(19) United States
(12) Patent Application Publication
(10) Pub. No.: US 2024/0024418 A1
(43) Pub. Date: Jan. 25, 2024

(54) METHOD FOR TREATING CANCER AND INFLAMMATORY DISEASES USING STEM CELL- DERIVED EXTRACELLULAR VESICLES COMPRISING SIRP

(71) Applicant: SHIFTBIO INC., Seoul (KR)

(72) Inventors: Gi-Hoon NAM, Seoul (KR); Gi Beom KIM, Seoul (KR); Seohyun KIM, Seoul (KR); Yoon Kyoung KIM, Seoul (KR); Jae Hyun KIM, Seoul (KR); Seonghyun KIM, Seoul (KR)

(21) Appl. No.: 18/357,160

(22) Filed: Jul. 24, 2023

Related U.S. Application Data
(60) Provisional application No. 63/391,774, filed on Jul. 24, 2022.

(51) Int. Cl. A61K 38/17; C12N 5/0775; C12N 15/86; A61K 47/69; A61P 29/00; A61P 1/16

(52) U.S. Cl. CPC A61K 38/1774; C12N 5/0663; C12N 15/86; A61K 47/6901; A61P 29/00; A61P 1/16; C12N 2510/02; C12N 2740/15043

(57) ABSTRACT

The present invention generally relates to a stem cell-derived extracellular vesicle (EV) comprising a signal regulatory protein (SIRP) and a method for preventing or treating cancer and/or inflammatory disease, condition, or symptom by using the stem cell-derived EV.

### CROSS-REFERENCE TO RELATED APPLICATION

The application claims priority to U.S. Application No. 63/391,774 filed on Jul. 24, 2022, which is incorporated herein by reference.

### REFERENCE TO ELECTRONIC SEQUENCING LISTING

The application contains a Sequence Listing which has been submitted electronically in .XML format and is hereby incorporated by reference in its entirety. Said .XML copy, created on Jul. 21, 2023, is named “Seq_SFT-P30003.xml” and is 10,893 bytes in size. The sequence listing contained in this .XML file is part of the specification and is hereby incorporated by reference herein in its entirety

### TECHNICAL FIELD

The present invention generally relates to a stem cell-derived extracellular vesicle (EV) comprising a signal regulatory protein (SIRP) and a method for preventing or treating cancer and/or inflammatory disease, condition, or symptom by using the stem cell-derived EV.

### BACKGROUND

The transmembrane protein CD47, ubiquitously expressed on the surfaces of various cell types including cancer cells, serves as a “don't-eat-me” signal. By interacting with signal-regulatory protein α (SIRPα) on macrophages, cells expressing CD47 can inhibit the phagocytic activity of these macrophages, thus evading immune surveillance. Disrupting the interaction between CD47 and SIRPα has emerged as a promising therapeutic strategy to surmount the CD47-mediated phagocytic barrier in cancer. Indeed, therapies involving anti-CD47 antibodies are presently under clinical investigation for multiple cancer types.

An innovative approach to obstructing the CD47-SIRPα interaction involves the use of engineered SIRP-EVs—nano-sized extracellular vesicles (50-150 nm) that display SIRPα on their surfaces. Compared to existing CD47 blockades, SIRP-EVs offer two primary advantages. First, their membrane organization offers a platform for SIRPα homodimerization, a process naturally occurring in macrophages, thereby allowing optimal protein functionality. Koh E, et al., Exosome-SIRPα, a CD47 blockade increases cancer cell phagocytosis. Biomaterials, 2017; 121: p. 121-129; Eunji C, et al., Comparison of exosomes and ferritin protein nanocages for the delivery of membrane protein therapeutics, 2018; 279:326-335, which are incorporated herein by reference. Moreover, unlike other CD47 blockades, such as CD47 antibodies and SIRPα-Fc fusion proteins, SIRP-EVs not only block overexpressed CD47 but also eliminate it through endocytosis-mediated clearance following binding. Kim Y K, et al., Advantage of extracellular vesicles in hindering the CD47 signal for cancer immunotherapy. J Control Release, 2022. 351:727-738, which is incorporated herein by reference. Preliminary results indicate that the EV modality possesses certain advantages over other competitive modalities in terms of CD47 blockade.

The inhibition of CD47-SIRPα interaction also shows promise in the treatment of chronic and acute inflammatory diseases. Fibrosis, a form of chronic inflammation, is characterized by the excessive accumulation of extracellular matrix (ECM) components, such as collagen and fibronectin, in damaged tissues. Fibrosis is a prevalent feature in numerous chronic inflammatory diseases, including end-stage liver disease, idiopathic pulmonary fibrosis, pulmonary artery hypertension, scleroderma, heart failure, chronic kidney disease, and rheumatoid arthritis. Wynn T A, Ramalingam T R, Mechanisms of fibrosis: therapeutic translation for fibrotic disease. Nat Med, 2012. 18(7): p. 1028-1040; Joel R, Edward M, Sonsoles P, Sergio A J, Human Fibrotic Diseases: Current Challenges in Fibrosis Research. Methods Mol Biol. 2017; 1627:1-23, which are incorporated herein by reference. As a pathological wound healing process, fibrosis results in the formation of permanent scar tissue, which can disrupt or completely inhibit the normal structure and function of the affected organ or tissue. Regrettably, the dearth of effective treatments for fibrosis can lead to fatal outcomes for patients.

Intriguingly, CD47 upregulation has been observed in pathological fibroblasts from several fibrotic diseases. These fibroblasts, due to their high CD47 expression, inhibit the phagocytic activity of macrophages. Gerlinde W, et al., Unifying mechanism for different fibrotic diseases. Proc Natl Acad Sci USA, 2017. 114(18):4757-4762, which are incorporated herein by reference. CD47 inhibition can stimulate the phagocytic removal of these pathological lung fibroblasts and activate the adaptive immune system, leading to clearance of lung fibrosis in a mouse model. Cui L, et al., Activation of JUN in fibroblasts promotes pro-fibrotic programme and modulates protective immunity. Nat Commun, 2020. 11(1): p. 2795, which is incorporated herein by reference. Furthermore, blocking CD47 can both prevent and reverse fibrotic skin changes in mouse models of scleroderma. Lerbs T, et al., CD47 prevents the elimination of diseased fibroblasts in scleroderma. JCI Insight, 2020. 5(16), which is incorporated herein by reference.

Necroptosis, a type of programmed cell death, plays a pivotal role in the pathogenesis of numerous inflammatory diseases, including but not limited to, neonatal hypoxia-ischemia brain injury, traumatic brain injury, various types of strokes, amyotrophic lateral sclerosis, neurodegenerative diseases, pulmonary and renal conditions, cardiac diseases, gastrointestinal disorders, and certain hepatic conditions. H Zhao, T Jaffer, S Eguchi, Z Wang, A Linkermann, D Ma, Role of necroptosis in the pathogenesis of solid organ injury. Cell Death Dis. 2015. 19;6(11), which is incorporated herein by reference. A noteworthy observation from prior studies is the upregulation of CD47 in necrotic cells, a phenomenon that inhibits their clearance.

Consequently, molecules leaking from these uncleared cells, known as damage-associated molecular patterns, aggravate inflammation in a self-perpetuating cycle, thereby exacerbating the disease state. Gerlach B D, et al., Resolvin D1 promotes the targeting and clearance of necroptotic cells. Cell Death Differ, 2020. 27(2): p. 525-539, which is incorporated herein by reference.

Therapeutic strategies involving anti-CD47 treatment have been proposed to stimulate the phagocytosis of these cells by macrophages, thereby curtailing this cycle. This approach has demonstrated promising effects in diseases such as atherosclerosis and vascular inflammation through the clearance of necrotic cores exhibiting high CD47 expression. Kojima Y, et al. CD47-blocking antibodies restore phagocytosis and prevent atherosclerosis. Nature. 2016 Aug. 4; 536(7614):86-90; Kai-Uwe J, et al., Effect of CD47 Blockade on Vascular Inflammation. N Engl J Med. 2021. 384(4):382-383, which are incorporated herein by references. In the context of non-alcoholic steatohepatitis (NASH), a chronic inflammatory disease witnessing a rapid surge in prevalence, necroptotic cells marked by CD47 overexpression have been frequently identified. Therapeutic intervention with anti-CD47 antibodies has shown potential to impede disease progression by partially curtailing the infiltration of immune cells into the liver and mitigating inflammation and fibrosis. Gwag T, Ma E, Zhou C, Wang S, Anti-CD47 antibody treatment attenuates liver inflammation and fibrosis in experimental non-alcoholic steatohepatitis models. Liver Int, 2022. 42(4): p. 829-841; Shi H, et al., CD47-SIRPα axis blockade in NASH promotes necroptotic hepatocyte clearance by liver macrophages and decreases hepatic fibrosis. Sci Transl Med, 2022. 23;14(672):eabp8309, which are incorporated herein by reference.

Significantly, in murine models of viral infections, a category of acute inflammatory diseases, CD47 expression was considerably augmented. Treatment with an anti-CD47 antibody in these cases activated macrophages, bolstered T-cell immune responses, and, collectively, decreased the viral load. Cham L B, et al., Immunotherapeutic Blockade of CD47 Inhibitory Signaling Enhances Innate and Adaptive Immune Responses to Viral Infection. Cell Rep, 2020. 31(2): p. 107494, which is incorporated herein by reference. Notably, the overexpression of CD47 has been implicated as a crucial factor in worsening the disease course in COVID-19 infections. McLaughlin K M, et al., A Potential Role of the CD47/SIRPalpha Axis in COVID-19 Pathogenesis. Curr Issues Mol Biol. 2021 22;43(3):1212-1225, which is incorporated herein by reference.

Taken together, these findings suggest that SIRP-EVs may demonstrate significant therapeutic efficacy in both acute and chronic inflammatory diseases, especially those characterized by pathological lesions with high CD47 expression. Stem cell-derived extracellular vesicles have already established their therapeutic potential in various inflammatory conditions. Harrell C R, et al., Mesenchymal Stem Cell-Derived Exosomes and Other Extracellular Vesicles as New Remedies in the Therapy of Inflammatory Diseases Cells. 2019 11;8(12):1605, which is incorporated herein by reference. Thus, engineering these vesicles to express SIRPα can potentially serve as an effective therapeutic strategy in inflammatory diseases typified by CD47 overexpression.

### SUMMARY

An aspect of the present invention provides a method for preventing or treating cancer or inflammatory disease, condition, or symptom, the method comprising administering to a subject in need thereof a prophylactically or therapeutically effective amount of stem cell-derived extracellular vesicles that comprise a signal regulatory protein (SIRP), a fragment of the SIRP, a variant of the SIRP, a fragment of the variant, and a variant of the fragment.

In some embodiments, the SIRP is SIRPα, SIRP7, or both. See U.S. Pat. No. 11,319,360, which is incorporated herein by reference.

In some embodiments, the stem cells are embryonic stem cells or adult stem cells.

In certain embodiments, the adult stem cells are selected from the group consisting of hematopoietic stem cells, mesenchymal stem cells, neural stem cells, human embryonic stem cells, induced pluripotent stem cells, human tissue-derived mesenchymal stromal cells, multipotent stem cells, cardiac stem cells, and amniotic epithelial cells.

In certain embodiments, the mesenchymal stem cells are derived from one or more tissues selected from the group consisting of umbilical cord, cord blood, bone marrow, fat, muscle, nerve, skin, amnion, and placenta.

In some embodiments, the SIRP, the fragment, or the variant is linked to at least one EV protein.

In certain embodiments, the EV protein is selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LlCAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment of the above-listed proteins, a variant of the above-listed proteins, a variant of the fragment, and a fragment of the variant.

In some embodiments, the cancer and inflammatory disease, condition, or symptom is characterized by overexpression of CD47.

In some embodiments, the inflammatory disease, condition, or symptom is acute or chronic inflammatory diseases, condition, or symptom.

In some embodiments, the inflammatory disease, condition, or symptom is selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, non-alcoholic steatohepatitis (NASH), inflammatory arthritis, gout, multiple sclerosis, psoriasis, acute respiratory distress syndrome (ARDS), diabetic ulcers, non-healing wounds, lupus, autoimmune diseases associated with acute or chronic inflammation, and acute or chronic inflammation associated with viral, bacterial or fungal infection.

In certain embodiments, the organ failure is selected from the group consisting of acute liver failure, acute renal failure, acute respiratory failure, acute heart failure, acute brain failure, and multiple organ failure.

In certain embodiments, the viral infection is selected from the group consisting of hepatitis virus infection, ZIKA virus infection, herpes virus infection, papillomavirus infection, influenza virus infection, coronavirus infection, COVID-19, and severe acute respiratory syndrome (SARS).

In certain embodiments, the fibrosis is selected from the group consisting of pulmonary fibrosis, cystic fibrosis, idiopathic pulmonary fibrosis, interstitial pulmonary fibrosis, liver fibrosis, bridging fibrosis of the liver, arthrofibrosis, keloid fibrosis, mediastinal fibrosis, myelofibrosis, myocardial fibrosis, nephrogenic systemic fibrosis, progressive massive fibrosis, retroperitoneal fibrosis, organ fibrosis, and stromal fibrosis.

In some embodiments, the cancer is selected from the group consisting of melanoma, renal cancer, prostate cancer, breast cancer, colon cancer and lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumor of childhood, lymphocytic lymphoma, cancer of the bladder, cancer of the kidney or ureter, carcinoma of the renal pelvis, neoplasm of the central nervous system (CNS), non-small cell lung cancer (NSCLC), primary CNS lymphoma, tumor angiogenesis, spinal axis tumor, brain stem glioma, pituitary adenoma, Kaposi's sarcoma, epidermoid cancer, squamous cell cancer, T-cell lymphoma, B-cell lymphomas, and environmentally induced cancer including cancer induced by asbestos (e.g., mesothelioma).

In some embodiments, the extracellular vesicle comprises an anti-cancer agent, an anti-inflammatory agent, or both.

The above and other aspects and embodiments of the present invention will be discussed in more detail below.

### BRIEF DESCRIPTION OF THE FIGURES

The figures depict various embodiments of the present invention for purposes of illustration only. One skilled in the art will readily recognize from the following discussion that alternative embodiments of the structures and methods illustrated herein may be employed without departing from the principles of the invention described herein.

FIG. 1 illustrates the DNA construct of some embodiments of the present invention, in which SIRPα represents a signal-regulatory protein alpha, ESM represents an extracellular vesicle sorting motif.

FIG. 2 illustrate the CD47 expression in tissues of various inflammatory diseases, fibrosis, NASH, acetaminophen (APAP) induced acute liver failure (ALF), ConA induced ALF and Cisplatin induced acute kidney injury (AKI), in accordance with the time and induction dose.

FIG. 3A illustrate comparison results of efficacy, which were demonstrated by liver tissue analysis, of engineered HEK293 cells derived SIRP-EV in mouse fibrosis models.

FIG. 3B illustrate comparison results of efficacy, which were demonstrated by liver tissue analysis, of engineered HEK293 cells derived SIRP-EV in mouse NASH models.

FIG. 4A to 4C illustrate comparison results of efficacy of engineered HEK293 cells derived SIRP-EV in LPS/D-galN induced mouse ALF model, live tissue macroscope, survival rate, biochemistry, cytokine analysis and liver immune cell analysis.

FIG. 5 illustrate antitumor effect compared with Anti-CD47 antibody and engineered HEK293 cells derived SIRP-EV.

FIG. 6A to 6D illustrate the results of proteomics of engineered mesenchymal stem cell (MSC) derived SIRP-EV (_{MSC}SIRP-EV) retaining MSC characteristics.

FIGS. 7A and 7B illustrate comparison results of SIRPα expression determined by the western blot data and superiority of _{MSC}SIRP-EV over other EV.

FIG. 8A to 8E illustrate comparison results of efficacy, which were demonstrated by survival, liver tissue macroscope and liver tissue damage analysis, of _{MSC}SIRP-EV in LPS/D-galN induced mouse ALF model.

FIG. 9A to 9D illustrate comparison results of efficacy, which were demonstrated by biochemistry, liver tissue immune cell analysis and cytokine analysis of FIG. 8.

FIG. 10 illustrate protective effect of _{MSC}SIRP-EV for kidney complications in FIG. 8.

FIG. 11A to 11C illustrate comparison results of efficacy, which were demonstrated by liver tissue macroscope, biochemistry and liver tissue damage analysis, of _{MSC}SIRP-EV in APAP induced mouse ALF model.

### DETAILED DESCRIPTION

### 1. Definition

Unless defined otherwise, all technical and scientific terms used herein have the meaning commonly understood by a person skilled in the art to which this invention belongs. As used herein, the following terms have the meanings ascribed to them below.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, the term “a combination thereof” or “combinations thereof” refers to all permutations and combinations of the listed items preceding the term. For example, “A, B, C, or a combination thereof” or “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “about” is used to indicate that a value includes the inherent variation of error for the composition, the method used to administer the composition, or the variation that exists among the study subjects.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally, extracellular vesicles range in diameter from 20 nm to 1000 nm, and can comprise various macromolecular cargo either within the internal space, displayed on the external surface of the extracellular vesicle, and/or spanning the membrane. The cargo can comprise small molecules, nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “exosome” refers to a cell-derived small vesicle comprising a membrane that encloses an internal space, and which is generated from said cell by direct plasma membrane budding or by fusion of the late endosome with the plasma membrane. The exosome comprises lipid or fatty acid and polypeptide and optionally comprises a therapeutic active payload, a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle.

As used herein, the terms “exosome protein,” “exosomal polypeptide,” “exosomal protein,” “EV polypeptide,” and “EV protein” are used interchangeably herein and shall be understood to relate to any protein or polypeptide that can be utilized to transport a polypeptide construct (which comprises, in addition to the exosome protein, SIRP) to an extracellular vesicle. More specifically, the term “exosome protein” shall be understood as comprising any protein or polypeptide that enables transporting, trafficking or shuttling of a polypeptide construct to an extracellular vesicle, such as an exosome. Examples of such exosome proteins are for instance CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LlCAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment thereof, a variant thereof, a variant of the fragment and a fragment of the variant and any combinations thereof, but numerous other polypeptides capable of transporting a polypeptide construct to EVs are comprised within the scope of the present invention. The exosome proteins are typically of human origin and can be found in various publicly available databases such as Uniprot, RCSB, etc.

As used herein, the term “SIRP” refers to a regulatory membrane glycoprotein that is expressed predominantly in bone marrow cells and expressed in stem cells or neurons. Four kinds of SIRPs, i.e., SIRPα, SIRPP, SIRP7 and SIRPS are reported up to date. Among them, SIRPα and SIRP7 are known to be inhibitory receptors and interact with the CD47 protein which is a transmembrane protein widely expressed in various cancer cells. The interaction between SIRP and CD47 is called the “don't eat me” signal. This interaction negatively regulates the effector function of innate immune cells, such as phagocytosis of tumor cells by them. This is similar to the self-signal provided by MHC I family molecules via Ig-like or Ly49 receptors. Cancer cells overexpressing CD47 activate SIRPα or SIRP7 to inhibit macrophage-mediated destruction. Recent studies have shown that high-affinity mutants of SIRPα increase the phagocytosis of cancer cells by masking CD47 on cancer cells. Weiskopf et al., Science 341 (6141): 88-91, 2013, which is incorporated herein by reference.

As used herein, the term “SIRPα” refers to a cell surface type I transmembrane protein that is expressed on macrophages and is member of the SIRP/SHPS (CD172) family within the Ig superfamily. SIRPα is a receptor for CD47. Other names in the art for SIRPα include: signal regulatory protein alpha, tyrosine-protein phosphatase non-receptor type substrate 1, BIT, CD172A, MFR, MYD-1, P84, PTPNS1, and SHPS1. An exemplary protein sequence for human SIRPα is GENBANK® Accession no. AAH33092.1 (sequence includes signal peptide), which is encoded by nucleic acid sequence GENBANK® Accession no. BC033092.1.

As used herein, “SIRP7” refers to a cell surface type I transmembrane protein that is another member of the SIRP/SHPS (CD172) family within the Ig superfamily and expressed, for instance, on T cells and activated NK cells. SIRP7 can bind CD47 but a signaling mechanism is not known. Other names in the art for SIRP7 include: signal regulatory protein gamma, and SIRP beta 2. An exemplary protein sequence for human SIRP7 is GENBANK® Accession no. NP_061026.2 (sequence includes signal peptide), which is encoded by nucleic acid sequence GENBANK® Accession no. NM_018556.3.

As used herein, the term “a fragment” of a protein, peptide, or nucleic acid refers to a segment of the protein, peptide, or nucleic acid. The fragments of the protein, peptide, or nucleic acid in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the protein, peptide, or nucleic acid.

As used herein, the term “variant” of a protein, peptide, or nucleic acid refers to a protein, peptide, or nucleic acid having has at least one amino acid or nucleotide which is different from the protein, peptide, or nucleic acid. A variant of a protein, peptide, or nucleic acid includes, but is not limited to, a substitution, deletion, frameshift, or rearrangement in the protein, peptide, or nucleic acid. The term may be used interchangeably with the term “mutant.” The variants of the protein, peptide, or nucleic acid in accordance with some embodiments of the present invention may retain at least 30%, 35%, 40%, 45%, 50%, 55%, 60%, 65%, 70%, 75%, 80%, 85%, 90%, 95%, 99%, or 100% of the ability of the protein, peptide, or nucleic acid.

As used herein, the term “stem cell-derived extracellular vesicle” refers to an extracellular vesicle that is derived from a stem cell. The stem cells may be embryonic stem cells or adult stem cells. The adult stem cells may comprise stem cells selected from the group consisting of mesenchymal stem cells, human tissue-derived mesenchymal stromal cells, human tissue-derived mesenchymal stem cells, multipotent stem cells, and amniotic epithelial cells. The mesenchymal stem cells may be derived from one or more tissues selected from the group consisting of umbilical cord, cord blood, bone marrow, fat, muscle, nerve, skin, amnion, and placenta.

As used herein, the term “producer cell” or “host cell” refers to a cell used for generating an extracellular vesicle. In the present invention, a producer cell is preferably a stem cell, and more preferably a mesenchymal stem cell (MSC). The producer cell may be transformed or transfected by one or more vectors that contain or contains exogenous sequence(s) or DNA construct(s). In some embodiments, the producer cell can be transformed or transfected by one single vector that contains an exogenous sequence or a DNA construct encoding a peptide comprising SIRP and exosome protein. In some embodiments, the producer cell can be transformed or transfected by one single vector that contains an exogenous sequence or a DNA construct encoding a peptide comprising SIRP and exosome protein and an exogenous sequence or a DNA construct encoding a therapeutically active payload. In some embodiments, the producer cell can be transformed or transfected by a vector that contains an exogenous sequence or a DNA construct encoding a peptide comprising STRP and exosome protein and another vector that contains an exogenous sequence or a DNA construct encoding a therapeutically active payload. In some embodiments, the producer cell can be transformed or transfected with at least one additional exogenous sequence or DNA construct encoding another protein or peptide (e.g., a targeting moiety). The additional exogenous sequence can be introduced into the vector that contains an exogenous sequence or a DNA construct encoding a peptide comprising SIRP and exosome protein, an exogenous sequence or a DNA construct encoding a therapeutically active payload, or both. In some embodiments, the exogenous sequence or DNA construct encoding a therapeutically active payload, the additional exogenous sequence or DNA construct encoding another protein or peptide, or both can be introduced into the producer cell so as to modulate endogenous gene expression of the producer cell. In some embodiments, the exogenous sequence or DNA construct encoding a therapeutically active payload, the additional exogenous or DNA construct sequence encoding another protein or peptide, or both can be introduced into the producer cell so as to produce the extracellular vesicle expressing SIRP that contains the therapeutically active payload, another protein or peptide, or both on the surface of the extracellular vesicle or in the extracellular vesicle.

As used herein, the term “therapeutically active payload” refers to a therapeutic agent capable of acting on a target that is contacted with an extracellular vesicle. In some embodiments, the therapeutically active payload can be introduced into an extracellular vesicle. In some embodiments, the therapeutically active payload can be introduced into a producer cell. Non-limiting examples of the therapeutically active payload include nucleotides, nucleic acids (e.g., DNA mRNA, miRNA, dsDNA, lncRNA, and siRNA), amino acids, polypeptides, lipids, carbohydrates, and small molecules.

As used herein, the term “linker” refers to any molecular structure that can conjugate a peptide or a protein to another molecule (e.g., a different peptide or protein, a small molecule, etc.). Suitable linkers are well known to those of skill in the art and include, but are not limited to, straight or branched-chain carbon linkers, heterocyclic carbon linkers, or peptide linkers (see, e.g., Chen et al., Advanced Drug Delivery Reviews, 2013, Vol. 65:10, pp. 1357-1369). The linkers can be joined to the carboxyl and amino terminal amino acids through their terminal carboxyl or amino groups or through their reactive side-chain groups. In addition, in some embodiments, linkers can be classified as flexible or rigid, and they can be cleavable (e.g., comprise one or more protease-cleavable sites, which can be located within the sequence of the linker or flanking the linker at either end of the linker sequence).

As used herein, the term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, the term “biologically active” refers to the ability to modify the physiological system of an organism without reference to how the active agent has its physiological effects.

As used herein, the terms “subject” and “patient” are used interchangeably herein and will be understood to encompass mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “administration” or “administering” of a composition refers to providing a composition to a subject in need of treatment. In accordance with embodiments of the present invention, therapeutic compositions may be administered singly or in combination with one or more additional therapeutic agents. The methods of administration of such compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration.

As used herein, the terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art. The compositions of the present disclosure may be administered by way of known pharmaceutical formulations, including tablets, pills, capsules, a liquid, an inhalant, a nasal spray solution, a suppository, a solution, a gel, an emulsion, an ointment, eye drops, ear drops, and the like.

As used herein, the term “therapeutically effective amount” refers to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a stem cell-derived exosome as disclosed herein required to provide a clinically significant decrease in disease symptoms. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower disease burden, the prophylactically effective amount in some aspects will be higher than the therapeutically effective amount.

### 2. Methods

In an aspect, the present invention provides a method for preventing or treating cancer or inflammatory disease, condition, or symptom. The method comprises administering a prophylactically or therapeutically effective amount of stem cell-derived extracellular vesicles (EVs) that comprise a signal regulatory protein (SIRP), a fragment of the SIRP, a variant of the SIRP, a fragment of the variant, or a variant of the fragment.

In some embodiments, the SIRP is SIRPα, SIRP7, or both.

In a preferred embodiment, the STRP is SIRPα, or a functional fragment thereof. Preferably, the functional fragment of SIRPα is the ectodomain of SIRPα, or a biologically active fragment thereof. As used herein, the term “biological active fragment thereof” in the context of the ectodomain of SIRPα encompasses any fragment of the ectodomain that can specifically bind to CD47 and inhibit CD47 binding to SIRPα, for example, on a macrophage or cancer cell.

SIRPα is a Type I membrane protein and has been sequenced in a number of species, including, but not limited to, mouse: GENBANK® Accession no. AAH62197.1; Human: GENBANK® Accession no. AAH33092.1; Pan troglodytes (chimpanzee): GENBANK® Accession no. JAA10535.1; Macaca mulatta (rhesus monkey): GENBANK® Accession no. AFE76783.1; Gorilla gorilla gorilla (Western lowland gorilla): GENBANK® Accession no. XP_004061735.1; and Bos taurus: GENBANK® Accession no. NP_786982.1.

In some embodiments, the STRP is a fragment of human SIRPα comprising at least 20, 30, 40, 50, 60, 70, 80, 90, 100, 110, 120, 130, 140, 150, 160, 170, 180, up to 508 contiguous amino acids of the full-length SIRPα protein, wherein the fragment specifically binds to CD47 and inhibits CD47 binding to SIRPα, for instance, on a macrophage or cancer cell.

In a preferred embodiment, the SIRPα is a human SIRPα. The human SIRPα has 503 amino acids. There are at least ten naturally occurring variants of wild-type human SIRPα.

In a preferred embodiment, the SIRPα ectodomain may be a D1 domain of SIRPα.

In a preferred embodiment, the SIRPα ectodomain may be a D1 domain of human SIRPα. The D1 domain of human SIRPα may be one of the D1 domain of variant human SIRPα. Exemplary sequences for the ectodomain of human SIRPα comprise or consist of SEQ ID Nos: 1-6.

SEQ ID NO 1:
EEELQVIQPDKSVLVAAGETATLRCTATSLIPVGPIQWERGAGPGRELI
YNQKEGHFPRVTTVSDLTKRNNMDFSIRIGNITPADAGTYYCVKFRKGS
PDDVEFKSGAGTELSVRAKP
SEQ ID NO 2
EEELQVIQPDKSVSVAAGESAILHCTVTSLIPVGPIQWERGAGPARELI
YNQKEGHFPRVTTVSESTKRENMDFSISISNITPADAGTYYCVKFRKGS
PDTEFKSGAGTELSVRAKP
SEQ ID NO 3:
EEELQVIQPDKSVLVAAGETATLRCTATSLIPVGPIQWERGAGPARELY
NQKEGHFPRVTTVSESTKRENMDFSISISNITPADAGTYYCVKFRKGSP
DTEFKSGAGTELSVRAKP
SEQ ID NO 4:
EEELQIIQPDKSVLVAAGETATLRCTITSLFPVGPIQWERGAGPGRVLI
YNQRQGPFPRVTTVSDTTKRNNMDFSIRIGNITPADAGTYYCIKFRKGS
PDDVEFKSGAGTELSVRAKP
SEQ ID NO 5:
EEELQIIQPDKSVSVAAGESAILHCTITSLFPVGPIQWFRGAGPARVLI
YNQRQGPFPRVTTVSETTKRENMDFSISISNITPADAGTYYCIKFRKGS
PDTEFKSGAGTELSVRAKP
SEQ ID NO 6:
EEELQIIQPDKSVLVAAGETATLRCTITSLFPVGPIQWERGAGPARVLI
YNQRQGPFPRVTTVSETTKRENMDFSISISNITPADAGTYYCIKFRKGS
PDTEFKSGAGTELSVRAKP

In some embodiments, the SIRP is a SIRP7, or a functional fragment thereof. Preferably, the functional fragment of SIRP7 is an ectodomain of SIRP7, or a biologically active fragment thereof. As used herein, the term “biological active fragment thereof” in the context of the ectodomain of SIRP7 encompasses any fragment of the ectodomain that can specifically bind to CD47 and inhibits CD47 binding to SIRP7, for example, on a macrophage or cancer cell. An exemplary sequence for an ectodomain of human SIRP7 is residues 26 to 357 of the polypeptide sequence of GENBANK® Accession No. NP_061026.2.

In a preferred embodiment, the SIRP7 is a human SIRPT.

An exemplary sequence for the ectodomain of human SIRP7 comprises or consists of: (SEQ ID NO: 7-9)

SEQ ID NO 7:
EEELQMIQPEKLLLVTVGKTATLHCTVTSLLPVGPVLWFRGVGPGRELI
YNQKEGHFPRVTTVSDLTKRNNMDFSIRISSITPADVGTYYCVKFRKGS
PENVEFKSGPGTEMALGAKP
SEQ ID NO 8:
EEELQIIQPEKLLLVTVGKTATLHCTVTSLFPVGPVLWFRGVGPGRVLI
YNQRQGPFPRVTTVSDTTKRNNMDFSIRISSITPADVGTYYCIKFRKGS
PENVEFKSGPGTEMALGAKP
SEQ ID NO 9:
EEELQIIQPEKLLLVTVGKTATLHCTITSLFPVGPVLWFRGVGPGRVLI
YNQRQGPFPRVTTVSDTTKRNNMDFSIRISSITPADVGTYYCIKFRKGS
PENVEFKSGPGTEMALGAKP

In some embodiments, the SIRP is a fragment of human SIRPγ comprising at least 20, 30, 40, 50, 60, 70, 80, 90, 100, 110, 120, 130, 140, 150, 160, 170, 180, up to 307 contiguous amino acids of the full-length SIRPγ protein, wherein the fragment specifically binds to CD47 and inhibits CD47 binding to SIRPγ, for instance, on a macrophage or cancer cell.

In some embodiments, the EVs may be surface-engineered EVs to comprise STRP, in order to enhance their therapeutic activity in various diseases mediated by CD47 such as cancer and inflammatory diseases. The term “surface-engineered EVs” refers to EVs with membrane modified in its composition. For example, the surface-engineered EVs may have a polypeptide construct on the surface of the EVs at a higher (or lower) density than a naturally occurring EVs do. In the present invention, a surface-engineered EVs can be produced from a genetically-engineered producer cell or a progeny thereof. For example, a surface-engineered EVs can be produced from stem cells transformed or transfected with an exogenous sequence or a DNA construct encoding the polypeptide construct comprising SIRP.

In some embodiments, the polypeptide construct comprising SIRP may further comprise at least one exosome protein, in order to drive the internalization into EVs of the polypeptide construct comprising STRP. Such exosome protein may be selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LlCAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment of the above-listed proteins, a variant of the above-listed proteins, a variant of the fragment, a fragment of the variant, and any combinations thereof, but not limited thereto. In the examples of the present invention, a fragment of PTGFRN (named as Extracellular Vesicle Sorting Motif (ESM)) was used as exosome protein.

In some embodiments, the polypeptide construct may be a fusion protein comprising the SIRP and at least one exosome protein. In some embodiments, the fusion protein may comprise SIRP linked (fused) directly or via a linker to at least one exosome protein.

In some embodiments, the linker may be a peptide linker. In some embodiments, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. In some embodiments, the peptide linker may be synthetic, i.e., non-naturally occurring. In some embodiments, a peptide linker may include peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in some embodiments the peptide linker can comprise non-naturally occurring polypeptides which are modified forms of naturally occurring polypeptides (e.g., comprising a mutation such as an addition, substitution, or deletion). Linkers can be susceptible to cleavage (“cleavable linker”) thereby facilitating release of the exogenous biologically active molecule. In some embodiments, the linker may comprise a non-cleavable linker.

In some embodiments, the STRP may be fused directly or via a linker to N-terminal, C-terminal or both of the exosome protein.

In a preferred embodiment, the SIRP may be fused directly or via a linker to N-terminal of the exosome protein.

In some embodiments, the polypeptide construct comprising SIRP and at least one exosome protein may be located or positioned in/on the membrane of EVs. In some embodiment, of the polypeptide construct comprising SIRP and at least one exosome protein, the exosome protein may be located or positioned in the membrane of EV at least in part. In some embodiment, of the polypeptide construct comprising SIRP and at least one exosome protein, the STRP may be located or positioned on the membrane of EVs. In some embodiment, of the polypeptide construct comprising SIRP and at least one exosome protein, the STRP may be expressed or displayed or presented on the membrane of EVs.

In some embodiments, the stem cells are human stem cells. In some embodiments, the stem cells are surface-engineered stem cells. In some embodiments, the stem cells are surface-engineered human stem cells. In some embodiments, the stem cells are selected from the group consisting of: adult stem cells, embryonic stem cells (ESCs), induced pluripotent stem cells, cord blood stem cells and amniotic fluid stem cells. In some embodiments, the adult stem cells are selected from the group consisting of mesenchymal stem cells, human tissue-derived mesenchymal stromal cells, human tissue-derived mesenchymal stem cells, multipotent stem cells, and amniotic epithelial cells. In some embodiments, the mesenchymal stem cells are derived from one or more tissues selected from the group consisting of umbilical cord, cord blood, bone marrow, fat, muscle, nerve, skin, amnion, and placenta. In some embodiments, the adult stem cells are selected from the group consisting of: neural stem cells, skin stem cells, epithelial stem cells, skeleton muscle satellite cells, mesenchymal stem cells, adipose-derived stem cells, endothelial stem cells, dental pulp stem cells, hematopoietic stem cells (including bone marrow stem cells, bone marrow mesenchymal stem cells, and the like) and placenta derived stem cells (including placenta derived mesenchymal stem cells, and the like).

According to the examples of the present invention, the choice of EV producer cell is a key to achieving superior therapeutic effect of EVs comprising SIRP. It has been demonstrated in the example of the present invention that EVs comprising SIRP (SIRP-EVs) derived from human bone marrow mesenchymal stem cells (_{MSC}SIRP-EVs) can synergize the regenerative potential inherited from stem cells and the pathogenic cell removal effect of the expressed SIRPα. Specifically, it has been shown that in acute organ injury models, _{MSC}SIRP-EVs could induce a stronger therapeutic effect compared to plain stem cell-derived EVs (_{M}scCon-EVs) and SIRP-EVs derived from HEK293 cells. Intriguingly, even at concentrations less than a twentieth of SIRP-EVs (HEK293 cell-derived), _{MSC}SIRP-EVs has been proven to generate sufficient therapeutic effects in acute inflammatory diseases.

In some embodiments, the EVs may further comprise at least one therapeutically active payload on the surface of the EVs, inside the EVs, or both. In some embodiments, the therapeutically active payload may be selected from the group consisting of nucleotides, amino acids, peptides, proteins lipids, carbohydrates, and small molecules, but not limited thereto. In some embodiments, non-limiting examples of other suitable therapeutically active payload includes pharmacologically active drugs and genetically active molecules, including anti-cancer agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, which are incorporated herein by reference. Suitable payloads further include toxins, and biological and chemical warfare agents, for example see Somani, S. M. (ed.), Chemical Warfare Agents, Academic Press, New York (1992)), which is incorporated herein by reference.

In some embodiments, the EVs may comprise an anti-cancer agent, an anti-inflammatory agent, or both.

In some embodiments, the exemplary anti-cancer agent includes, but are not limited to, curcumin, interferons, cytokines (e.g., tumor necrosis factor, interferon α, interferon γ), antibodies (e.g. Herceptin (trastuzumab), T-DM1, AVASTIN (bevacizumab), ERBITUX (cetuximab), Vectibix (panitumumab), Rituxan (rituximab), and Bexxar (tositumomab)), anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DI-FR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin A analogs, Vitamin D3 analogs (e.g. EB 1089, CB 1093, and KH 1060), vitamin K, isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (TRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In some embodiments, the exemplary anti-inflammatory agent includes, but are not limited to curcumin, non-steroidal anti-inflammatory drugs (NSAIDs) including, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium. In addition, cytokine antagonists, such as aptamers of IL-10, IL-6, IL-8, TNF-alpha (TNF-α), IL-5, IL-13, TGF-beta (TGF-β), VEGF and etc., may be used for anti-inflammatory effects.

In some embodiments, the EVs may further comprise at least one targeting moiety. In some embodiments, the targeting moiety can be used for targeting the EVs to a specific organ, tissue, or cell for a treatment using the EVs. In certain embodiments, the targeting moiety may bind to a marker (or target molecules) expressed on a cell or a population of cells. In certain embodiments, the marker may be expressed on multiple cell types, e.g., all antigen-present cells (e.g., dendritic cells, macrophages, and B lymphocytes). In some embodiments, the marker may be expressed only on a specific population of cells (e.g., dendritic cells). Non-limiting examples of markers that are expressed on specific population of cells (e.g., dendritic cells) include a C-type lectin domain family 9 member A (CLEC9A) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), and any combination thereof. In some embodiments, the targeting moiety may be an antibody or antigen-binding fragment thereof. Antibodies and antigen-binding fragments thereof include whole antibodies, polyclonal, monoclonal and recombinant antibodies, fragments thereof, and they may further include single-chain antibodies, humanized antibodies, murine antibodies, chimeric, mouse-human, mouse-primate, primate-human monoclonal antibodies, anti-idiotype antibodies, antibody fragments (e.g., scFv, (scFv)2, Fab, Fab′, and F(ab′)2, F(abl)2, Fv, dAb, and Fd fragments), diabodies, and antibody-related polypeptides. Antibodies and antigen-binding fragments thereof may include bispecific antibodies and multispecific antibodies so long as they exhibit the desired biological activity or function.

In some embodiments, the cancer and inflammatory disease, condition, or symptom are CD47 positive. In some embodiments, the cancer and inflammatory disease, condition, or symptom are related to the overexpression of CD47.

As used herein, the term “CD47 positive” is used with reference to the phenotype of cells targeted by the EVs comprising SIRP. Cells that are CD47 positive can be identified by flow cytometry using CD47 antibody as the affinity ligand. CD47 antibodies that are labeled appropriately are available commercially for this use. In some embodiments, the CD47 positive disease cells of particular interest as targets for therapy with the EVs comprising SIRP are those that overexpressed CD47. These CD47 positive or CD47 overexpressed cells typically are disease cells, and present CD47 at a density on their surface that exceeds the normal CD47 density for a cell of a given type. CD47 overexpression will vary across different cell types but is meant herein to refer to any CD47 level that is determined, for instance by flow cytometry or by immunostaining or by gene expression analysis or the like, to be greater than the level measurable on a healthy counterpart cell having a CD47 phenotype that is normal for that cell type.

In some embodiments, the inflammatory disease, condition, or symptom is related to acute and/or chronic disease, condition, or symptom selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, dermatologic inflammation, gastrointestinal inflammation, tendinopathies, allergy, asthma, glomerulonephritis, pancreatitis, hepatitis, non-alcoholic steatohepatitis (NASH), gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, crohn's and ulcerative colitis, terminal ileitis, ophthalmic inflammation, retinal degeneration, retinal detachment, retinitis pigmentosa, inherited retinal diseases, age-related macular degeneration, glaucoma, inflammatory arthritis, rheumatoid arthritis, osteoarthritis, alcoholic steatohepatitis, hepatotoxicity, liver infection, remote liver injury, lupus, autoimmune diseases associated with acute or chronic inflammation, and acute or chronic inflammation associated with viral, bacterial or fungal infection, but not limited thereto (Gwag T, et al., Anti-CD47 antibody treatment attenuates liver inflammation and fibrosis in experimental non-alcoholic steatohepatitis models. Liver Int, 2022. 42(4): p. 829-841; Cui L, et al., Activation of JUN in fibroblasts promotes pro-fibrotic programme and modulates protective immunity. Nat Commun, 2020. 11(1): p. 2795; Lerbs T, et al., CD47 prevents the elimination of diseased fibroblasts in scleroderma. JCI Insight, 2020. 5(16); Gerlinde W, et al., Unifying mechanism for different fibrotic diseases. Proc Natl Acad Sci USA, 2017. 114(18):4757-4762; Kojima Y, et al. CD47-blocking antibodies restore phagocytosis and prevent atherosclerosis. Nature. 2016. 536(7614):86-90; Kai-Uwe J, et al., Effect of CD47 Blockade on Vascular Inflammation. N Engl J Med. 2021. 384(4):382-383; Yan T, et al., Necroptosis and Neuroinflammation in Retinal Degeneration. Front Neurosci. 2022. 16:911430; Qian W, et al., Dysregulated integrin αVβ3 and CD47 signaling promotes joint inflammation, cartilage breakdown, and progression of osteoarthritis. JCI Insight. 2019. 4(18):e128616; Cham L B, et al. Immunotherapeutic Blockade of CD47 Inhibitory Signaling Enhances Innate and Adaptive Immune Responses to Viral Infection. Cell Rep. 2020. 31(2):107494; McLaughlin K M, et al., A Potential Role of the CD47/SIRPalpha Axis in COVID-19 Pathogenesis. Curr Issues Mol Biol. 2021 22;43(3):1212-1225; H Zhao, et al., Role of necroptosis in the pathogenesis of solid organ injury. Cell Death Dis. 2015. 19;6(11); M Deutsch, et al., Divergent effects of RIP1 or RIP3 blockade in murine models of acute liver injury. Cell Death Dis. 2015. 6(5):e1759, which are incorporated herein by reference).

In certain embodiments, the organ failure is selected from the group consisting of acute liver failure, bone marrow failure, acute kidney failure, and acute heart failure, but not limited thereto.

In certain embodiments, the viral infection is selected from the group consisting of hepatitis virus infection, ZIKA virus infection, herpes virus infection, papillomavirus infection, influenza virus infection, coronavirus infection, COVID-19, and SARS, but not limited thereto.

In certain embodiments, the fibrosis is selected from the group consisting of pulmonary fibrosis, cystic fibrosis, idiopathic pulmonary fibrosis, skin fibrosis, kidney fibrosis, bone marrow fibrosis, interstitial pulmonary fibrosis, liver fibrosis, bridging fibrosis of the liver, arthrofibrosis, keloid fibrosis, mediastinal fibrosis, myelofibrosis, myocardial fibrosis, nephrogenic systemic fibrosis, progressive massive fibrosis, retroperitoneal fibrosis, and stromal fibrosis, but not limited thereto.

In some embodiments, the cancer includes particularly CD47 positive cancer or CD47 overexpressed cancer, including solid tumor. In some embodiments, the solid tumor includes CD47 positive or CD47 overexpressed tumor in bladder, brain, breast, lung, colon, ovary, prostate, liver, and other tissues as well.

In some embodiments, the cancer includes particularly CD47 positive cancer or CD47 overexpressed cancer, including liquid tumor. The “liquid tumor” is used interchangeably herein with “hematological cancer.” As used herein, the term “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, cutaneous T cell lymphoma (CTCL), Burkitt's lymphoma, Mantle cell lymphoma (MCL) and follicular lymphoma (small cell and large cell), among others. Myelomas include multiple myeloma (MM), giant cell myeloma, heavy-chain myeloma, and light chain myeloma and Bence-Jones myeloma. In some embodiments, the hematological cancer is a CD47 positive or CD47 overexpressed leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia. In other embodiments, the hematological cancer is a CD47 positive or CD47 overexpressed lymphoma or myeloma selected from Hodgkin's lymphoma, both indolent and aggressive non-Hodgkin's lymphoma, diffuse large cell lymphoma (DLBCL), mantle cell lymphoma, T cell lymphoma including mycosis fungoides, Sezary's syndrome, Burkitt's lymphoma, follicular lymphoma (small cell and large cell), multiple myeloma (MM), giant cell myeloma, heavy-chain myeloma, and light chain or Bence-Jones myeloma as well as leiomyosarcoma.

In some embodiments, the cancer is selected from the group consisting of melanoma, renal cancer, prostate cancer, breast cancer, colon cancer, rectum adenocarcinoma, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumor of childhood, lymphocytic lymphoma, cancer of the bladder, cancer of the kidney or ureter, carcinoma of the renal pelvis, neoplasm of the central nervous system (CNS), non-small cell lung cancer (NSCLC), primary CNS lymphoma, tumor angiogenesis, spinal axis tumor, brain stem glioma, glioblastoma multiforme, low-grade gliomas, pituitary adenoma, Kaposi's sarcoma, epidermoid cancer, squamous cell cancer, T-cell lymphoma, B-cell lymphomas, cholangiocarcinoma, thymoma, adrenocortical cancer, cervical cancer, endocervical cancer, myxofibrosarcoma, undifferentiated pleomorphic sarcoma, dedifferentiated liposarcoma, dysembryoplastic neuroepithelial tumor, ependymoma, nasopharyngeal carcinoma, choroid plexus carcinoma, myoepithelial carcinoma, alveolar rhabdomyosarcoma, rhabdomyosarcoma, atypical teratoid/mabdoid tumor, desmoplastic small round cell tumor, fibromatosis, synovial sarcoma, wilms tumor, myofibromatosis, ewing sarcoma, infantile fibrosarcoma, INI-deficient soft tissue sarcoma, medulloblastoma. and environmentally induced cancer including cancer e induced by asbestos (e.g., mesothelioma), but not limited thereto (Willingham S B, et al., The CD47-signal regulatory protein alpha (SIRPα) interaction is a therapeutic target for human solid tumors. Proc Natl Acad Sci USA. 2012. 109(17):6662-6667; Candas-Green D, et al. Dual blockade of CD47 and HER2 eliminates radioresistant breast cancer cells. Nat Commun. 2020. 11(1):4591; Yu L, Ding Y, Wan T, Deng T, Huang H, Liu J. Significance of CD47 and Its Association With Tumor Immune Microenvironment Heterogeneity in Ovarian Cancer. Front Immunol. 2021. 12:768115; Gupta A, Taslim C, Tullius B P, Cripe T P. Therapeutic modulation of the CD47-SIRPα axis in the pediatric tumor microenvironment: working up an appetite. Cancer Drug Resist. 2020. 3(3):550-562; Huang J, et al., Role of CD47 in tumor immunity: a potential target for combination therapy. Sci Rep. 2022. 12(1):9803, which are incorporated herein by reference).

### 3. Compositions

In another aspect, the present invention provides a pharmaceutical composition for preventing or treating cancer or inflammatory disease, condition, or symptom. The composition comprises stem cell-derived extracellular vesicles that comprise a signal regulatory protein (SIRP), a fragment of the SIRP, a variant of the SIRP, a fragment of the variant, or a variant of the fragment. The composition may further comprise a pharmaceutically acceptable carrier and/or excipient. Pharmaceutically acceptable excipients or carriers can be determined in part by the particular composition being administered, as well as by the particular method used to administer the composition. Accordingly, there is a wide variety of suitable formulations of pharmaceutical compositions. See, e.g., Remington's Pharmaceutical Sciences, Mack Publishing Co., Easton, Pa. 21st ed. (2005), which is incorporated herein by reference. The pharmaceutical compositions can be generally formulated sterile and in full compliance with all Good Manufacturing Practice (GMP) regulations of the U.S. Food and Drug Administration.

In some embodiments, a pharmaceutically acceptable carrier may be various oral or parenteral formulations. For the preparation of formulations, a diluent or excipient such as a filler, an extender, a binder, a humectant, a disintegrant, a surfactant, etc., may be used. Solid formulations for oral administration may include tablets, pills, powders, granules, capsules, etc., and these solid formulations may be prepared by adding at least one excipient, e.g., starch, calcium carbonate, sucrose or lactose, gelatin, etc. Additionally, lubricants, such as magnesium stearate, talc, etc., may be used, in addition to the simple excipient. Liquid formulations for oral administration may include suspensions, liquid medicines for internal use, emulsions, syrups, etc., and various excipients such as humectants, sweeteners, fragrances, and preservatives, may be used, in addition to the frequently used simple diluents such as water and liquid paraffin. Formulations for parenteral administration may include sterile aqueous solutions, non-aqueous solvents, suspensions, emulsions, lyophilized formulations, suppositories, etc. Examples of the non-aqueous solvents and suspensions may include vegetable oils such as propylene glycol, polyethylene glycol, and olive oil; an injectable ester such as ethyl oleate; etc. Examples of the bases for suppositories may include Witepsol, macrogol, Tween 61, cacao butter, laurinum, glycerogelatin, etc.

In some embodiments, the pharmaceutical composition may have one formulation selected from the group consisting of tablets, pills, powders, granules, capsules, suspensions, solutions, emulsions, syrups, sterilized aqueous solutions, non-aqueous solutions, lyophilized formulations, and suppositories.

In some embodiments, the pharmaceutical composition may be administered orally or parenterally. When administered parenterally, the pharmaceutical composition may be administered via various routes, including intravenous administration, intra-arterial administration, epidural administration, intracerebral administration, intracerebroventricular administration, nasal administration, intramuscular administration, intraperitoneal administration, subcutaneous administration, intradermal administration, transdermal absorption, etc.

In some embodiments, the pharmaceutical composition may be administered in a therapeutically effective amount.

In some embodiments, the pharmaceutical composition may be administered as an individual therapeutic agent, in combination with other therapeutic agents for cancer, or inflammatory diseases, or sequentially or simultaneously with a conventional therapeutic agent(s) and may be administered once or multiple times. It is important to administer an amount to obtain the maximum effect with a minimum amount without adverse effects considering all of the factors, and these factors can easily be determined by one of ordinary skill in the art.

Hereinafter, embodiments of the present disclosure will be described in detail with the following examples. However, the present disclosure is not limited to the examples explained. Rather, the examples are provided to sufficiently transfer the concept of the present disclosure to a person skilled in the art to thorough and complete contents introduced herein.

### Example 1

### Construction of Plasmid DNAs

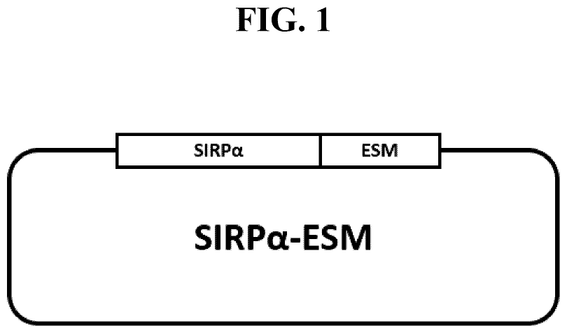

In accordance with one embodiment of the present invention, a DNA construct that can effectively translocate SIRPα protein onto the EV membrane was constructed. The commercial plasmid DNA was purchased from Origene, and an additional vector construct for the desired plasmid DNA sequence was acquired through gene synthesis service (Cosmo Genetech Co.). The vector was based on pcDNA3.1, retroviral vector, or lentiviral vector. The plasmid map of the constructed STRP-EV is shown in FIG. 1. As a control group, an empty vector (pcDNA3.1 or retroviral vector) was used.

To maximize the efficiency of the existing construct, the SIRPα-ESM construct was prepared. Through the sequence named Extracellular Vesicle Sorting Motif (ESM), the expression efficiency of the protein of desire to display on the EVs was maximized. The sequence of the ESM and the method of making the sequence are described in U.S. application Ser. No. 18/322,723, which is incorporated herein by reference.

SIRPα-ESM Sequence (SEQ ID NO: 10):
MMLQHLVIFCLGLVVONFCSPGSEEELQIIQPDKSVLVAAGETATLRCT
ITSLFPVGPIQWFRGAGPGRVLIYNQRQGPFPRVTTVSDTTKRNNMDFS
IRIGNITPADAGTYYCIKFRKGSPDDVEFKSGAGTELSVRAKPEFKYPL
LIGIGLSAVIGLLSCLIGYCSS

The above-mentioned plasmids were amplified and isolated according to a protocol of the Qiagen® Plasmid Maxi kit. More specifically, 1 μl (0.1 μg) of the plasmid DNA and 100 μl competent cells DH5a were mixed in a 1.5 ml microcentrifuge tube. Plasmid DNA was introduced to competent cells DH5a by heat shock. To elaborate, the microcentrifuge tube containing the mixture of plasmid DNA and competent cells DH5a was heated at 42° C. for 45 seconds using a heat block. The heated microcentrifuge tube was placed on ice for 2 minutes. After cooling down, 900 μl antibiotic-free room temperature LB agar media was added to the microcentrifuge tube. Then, this microcentrifuge tube was incubated at 37° C. for 45 minutes on a 200-rpm shaker. After incubation, 100 μl from the microcentrifuge tube was spread onto LB media containing plates with 100 μg/ml ampicillin. All plates were incubated overnight at 37° C. On the following day, a colony was taken from the surface of the plate and incubated in 2-3 ml of LB media with 100 μg/ml ampicillin at 37° C. for 8 hours. After incubation, 1 ml from the mixture of colony and LB media with antibiotics was transferred to a flask containing 500 ml of LB/ampicillin media and incubated overnight at 37° C. The bacterial cells were harvested by centrifugation at 6000×g for 15 min at 4° C., and the bacterial pellet was resuspended in Buffer P1 with RNase A 100 μg/ml. Buffer P2 was added and mixed thoroughly by vigorously inverting the sealed tube 4-6 times, and the resulting mixture was incubated at room temperature for 5 min. Chilled Buffer P3 was added and mixed immediately and thoroughly by vigorously inverting 4-6 times, and the resulting mixture was incubated on ice for 20 min. After centrifuging at ≥20,000×g for 30 min at 4° C., supernatant containing plasmid DNA was collected promptly. After centrifuging the supernatant again at ≥20,000×g for 15 min at 4° C., supernatant containing plasmid DNA was collected promptly. After equilibrating a QIAGEN-tip 500 by applying Buffer QBT and allowing the column to empty by gravity flow, the collected supernatant was applied to the QIAGEN-tip and allowed to enter the resin by gravity flow. After washing the QIAGEN-tip with Buffer QC, DNAs were eluted with Buffer QC. The eluted DNAs were precipitated by adding room-temperature isopropanol to the eluted DNA. After mixing and centrifuging immediately at ≥15,000×g for 30 min at 4° C., the supernatant was carefully decanted. After washing DNA pellet with room-temperature 70% ethanol and centrifuging at ≥15,000×g for 10 min, the supernatant was carefully decanted without disturbing the pellet. After air-drying the pellet for 5-10 min, the final plasmid DNAs were redissolved in a suitable volume of buffer.

### Example 2

### Isolation of EVs

To generate SIRP-EVs from engineered HEK293 cells, various HEK293 cell lines (either the original or its derivatives) were cultured at 37° C. with 5% CO₂ in Dulbecco's Modified Eagle's Medium (DMEM) supplemented with 10% fetal bovine serum (FBS). When cells reached a confluency of approximately 80% to 90%, they were transfected with specific plasmid DNA using suitable transfection reagents. Alternatively, for the establishment of stable cell lines, cells were infected with either retrovirus or lentivirus.

In case of transient transfection, the cells were transfected using transfection agents, such as lipofectamine 2000, lipofectamine 3000, or polyethylenimine (PEI). Cell medium was replaced with DMEM, and mixture of DNA and transfection reagent was added into the cells. The cells were then incubated at 37° C. with 5% CO2 for 24 hours. 24 hours post transfection, the medium containing transfection agents and plasmids was replaced with DMEM to which 10% FBS and 1% antibiotic-antimycotic were added. The transient transfected cells were incubated at 37° C. with 5% CO₂ for 24 hours. See, e.g., Gi Kim et al., Xenogenization of tumor cells by fusogenic exosomes in tumor microenvironment ignites and propagates antitumor immunity, SCIENCE ADVANCES, Vol 6, Issue 27 (Jul. 1, 2020), which is incorporated herein by reference.

In case of stable cell generation using a retrovirus, Plat-E cells were used to produce retrovirus packaging a retroviral vector containing a DNA sequence of interest and a DNA sequence of puromycin-resistance gene. More particularly, Plat-E cells were incubated at 37° C. with 50% CO₂ in DMEM to which 10% FBS was added. At the time when it had 80˜ 90% of confluency, the cells were transfected by the retroviral vector encoding a DNA sequence of interest by using lipofectamine 2000. After 24 hours, culture medium was replaced with DMEM supplemented 10% FBS and incubated for additional 24 hours. When 48 hours passed after transfection was made, culture medium containing viral particles was collected, centrifugated at 3,000 rpm, filtered with 0.45 μm filter, and used for HEK293 cell lines infection. See, e.g., Park, S Y, Yun, Y, Lim, J S. et al. Stabilin-2 modulates the efficiency of myoblast fusion during myogenic differentiation and muscle regeneration. Nat Commun 7, 10871 (2016), which is incorporated herein by reference.

To generate SIRP-EVs from engineered stem cells, lentiviral particles carrying the desired DNA sequence were obtained from Genscript and Flash Therapeutics. The optimal multiplicity of infection (MOI) for the lentivirus was established via a titration study. Subsequently, human bone marrow-derived mesenchymal stem cells (hBM-MSCs, sourced from RoosterBio) were transduced with the lentiviral particles using the RoosterGEM system (Roosterbio, catalog number: M40200) for a period of 24 hours. This process led to the creation of a stable cell line that produces SIRP-EVs.

To isolate EVs, culture medium from transiently transfected cells or stably expressing cells was replaced with DMEM medium supplemented with insulin-transferrin-selenium (Gibco) or EV collection media (RoosterCollect™-EV, supplied by RoosterBio). The cells were then incubated at 37° C. with 5% CO₂ for 48 hours. Post incubation, the cell supernatants were collected and subjected to a sequential centrifugation process: initially at 300 g for 10 minutes, followed by 2,000 g for 10 minutes, and finally 10,000 g for 30 minutes. The supernatants were subsequently filtered and concentrated using a tangential flow filtration (TFF) system or a 100 kDa Amicon Ultra-15 centrifugal filter unit. Following concentration, the supernatants were ultracentrifuged at 150,000 g for 1.5 hours to pellet the EVs. The resulting EV pellets were resuspended in phosphate-buffered saline (PBS) and stored at 4° C. or −80° C. until further use. See, e.g., Gi Kim et al., Xenogenization of tumor cells by fusogenic exosomes in tumor microenvironment ignites and propagates antitumor immunity, SCIENCE ADVANCES, Vol 6, Issue 27 (Jul. 1, 2020), which is incorporated herein by reference.

### Example 3

### CD47 as an Effective Target in Inflammatory Disease Models

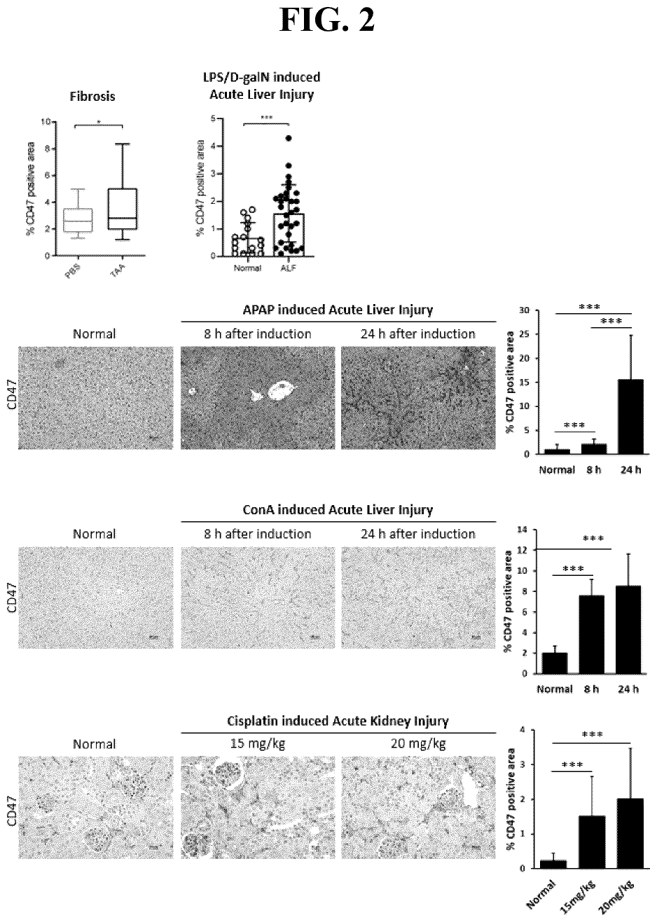

To confirm whether CD47 was overexpressed in chronic inflammatory diseases compared to normal tissues, a representative fibrosis model was established. Thioacetamide (TAA) was administered intraperitoneally at a dosage of 100 mg/kg three times a week for a total of 8 weeks (24 total injections) in 6-week-old C57BL/6 mice. Three days after the final TAA injection, liver tissue was extracted, and paraffin blocks were made. As a control, the liver from a healthy mouse of the same age was also made into a paraffin block. The liver tissue paraffin blocks were sectioned, fixed with acetone, and blocked with 3% H₂O₂ for 15 minutes to remove endogenous peroxidase activity. A pressure cooker was used for antigen retrieval, and after a 15-minute blocking process, the tissues were stained at room temperature for 1 hour using a CD47 (GTX 53912) antibody (1:500). Peroxidase substrates were used for detection, and microscope images were taken and quantified for the level of CD47 expression within the image field using ImageJ software (National Institute of Health, USA). As shown in FIG. 2, it was confirmed that the expression of CD47 was higher in tissues that induced fibrosis through TAA injection compared to normal liver tissues.

To confirm whether CD47 was overexpressed in acute inflammatory diseases compared to normal tissues, acute inflammatory disease models were established using Lipopolysaccharide (LPS)/D-Galactosamine (D-galN), acetaminophen (APAP), concanavalin A (Con A), and cisplatin. To create an LPS/D-galN induced acute liver injury model, LPS (10 μg/kg) and D-galN (700 mg/kg) were intraperitoneally (IP) injected into 7-week-old male C57BL/6 mice. For the APAP induced acute liver injury model, APAP (300 mg/kg) was IP injected into 7-week-old male C57BL/6 mice. For the Con A induced acute liver injury model, ConA (30 mg/kg) was intravenously injected into 7-week-old male C57BL/6 mice. To create the cisplatin induced acute kidney injury model, cisplatin (15 mg/kg or 20 mg/kg) was IP injected into 7-week-old male C57BL/6 mice.

To ascertain whether CD47 was overexpressed in acute inflammatory disease tissues compared to normal tissues, liver or kidney tissues were extracted and made into paraffin blocks 24 hours after LPS/D-galN injection, 8 or 24 hours after APAP injection, 8 or 24 hours after Con A injection, and 72 hours after cisplatin injection. As a control, the liver or kidney of a healthy mouse of the same age was also made into a paraffin block. The paraffin blocks were sectioned, fixed with acetone, and blocked with 3% H₂O₂ for 15 minutes to remove endogenous peroxidase activity. A pressure cooker was used for antigen retrieval, and after a 15-minute blocking process, the tissues were stained at room temperature for 1 hour using a CD47 (GTX 53912) antibody (1:500). Peroxidase substrates were used for detection, and microscope images were taken and quantified for the level of CD47 expression within the image field using ImageJ software (National Institute of Health, USA). As shown in FIG. 2, it was confirmed that the expression of CD47 was higher in tissues that induced acute inflammatory disease compared to normal tissues.

### Example 4

### Promising Efficacy of SIRP-EV in CD47 Overexpressed Diseases

The antifibrotic efficacy of SIRP-EV was evaluated in a chronic inflammatory disease model, fibrosis, where CD47 was overexpressed. A stable cell capable of producing SIRP-EV was created in HEK293 cell lines using a retrovirus, and SIRP-EV was obtained and used for the experiment through this.

After injecting 100 mg/kg TAA into the aforementioned fibrosis model for six weeks, SIRP-EV was injected at 50 μg (2.5 mg/kg) twice a week for three weeks, a total of six IV injections. To evaluate the efficacy of SIRP-EV in non-alcoholic steatohepatitis (NASH), known to be associated with fibrosis, 200 μg of streptozotocin was subcutaneously injected into male C57BL/6 mice two days after birth. From birth to the fourth week, a high-fat diet (57 kcal % fat) was fed. Starting from the sixth week, SIRP-EV or Con-EV was injected at 40 μg (2 mg/kg) twice a week for three weeks, a total of six IV injections. Three days after the last injection, the mice were sacrificed, and liver tissue was extracted to create paraffin blocks. As a control, the liver of a healthy mouse of the same age was also made into a paraffin block.

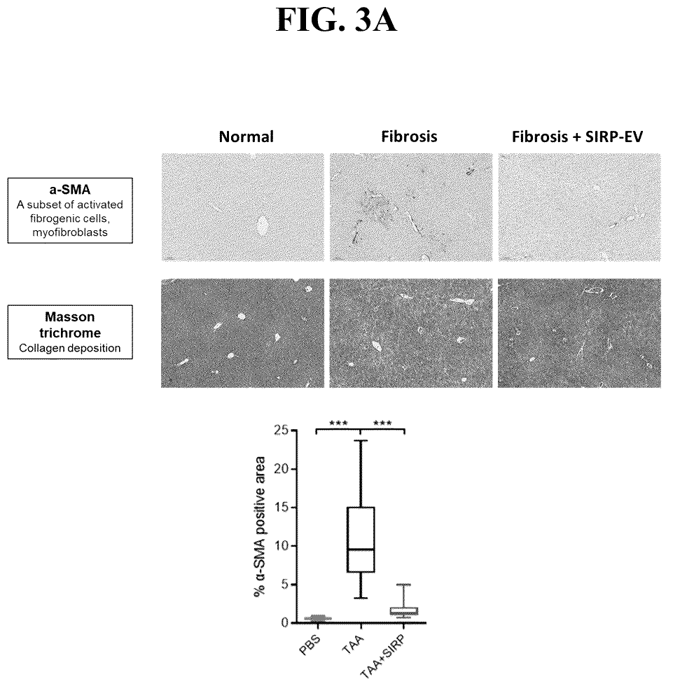

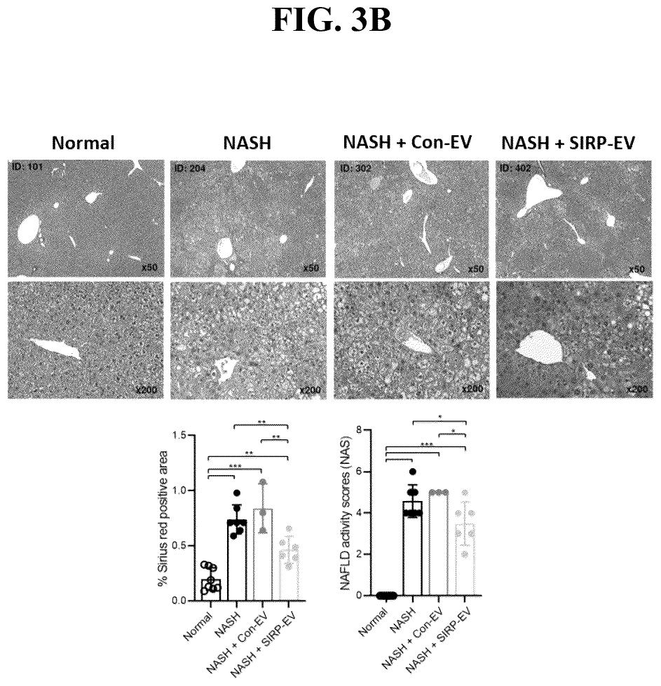

Slides was created from blocks of three groups: 1) normal mouse liver tissue, 2) liver tissue that induced fibrosis, and 3) liver tissue that induced fibrosis and was treated with SIRP-EV a-SMA staining (staining cells promoting fibrosis), and masson's trichrome staining (staining accumulated collagen) were performed to measure the degree of fibrosis. After sectioning the frozen block and fixing it with acetone, it was blocked with 3% H₂O₂ for 15 minutes to remove endogenous peroxidase activity. A pressure cooker was used for antigen retrieval, and after a 15-minute blocking process, the tissues were stained at 37° C. for 45 minutes using an alpha-SMA (NBP1-30894) antibody (1:2000). Peroxidase substrates were used for detection, and microscope images were taken and quantified for the level of alpha-SMA expression within the image field using ImageJ software (National Institute of Health, USA). Collagen deposition was visualized through a Masson's trichrome staining assay performed on paraffin-embedded tissue blocks. As demonstrated in FIG. 3A, SIRP-EV treatment significantly reduced both alpha-SMA-positive regions and collagen deposition in fibrotic liver tissue. As shown in FIG. 3B, the efficacy of SIRP-EV was confirmed that collagen accumulation and NAS score (NAFLD activity score) were decreased in the SIRP-EV treated group. SIRP-EV demonstrated significant inhibition of fibrosis in both liver fibrosis and NASH models. These findings imply that, in conditions such as fibrosis and NASH characterized by high CD47 expression, SIRP-EV could potentially lead to enhanced therapeutic outcomes.

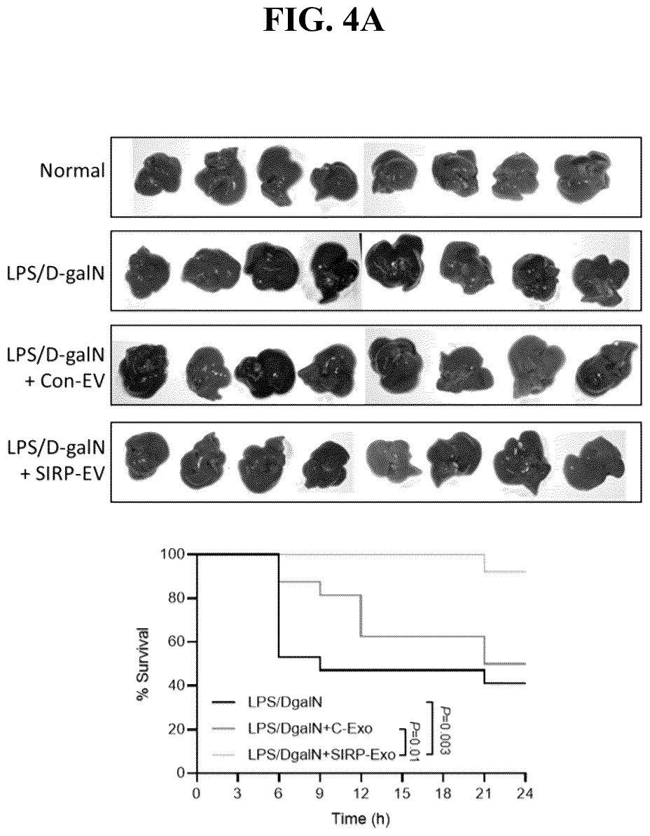

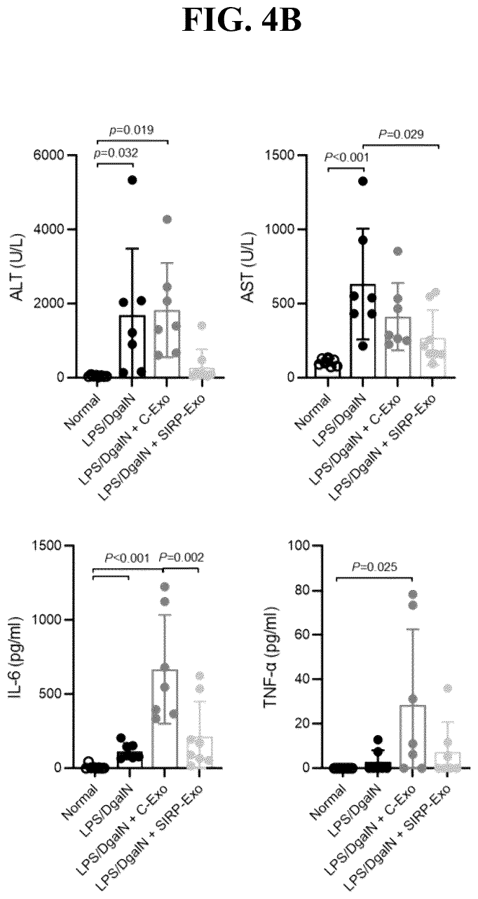

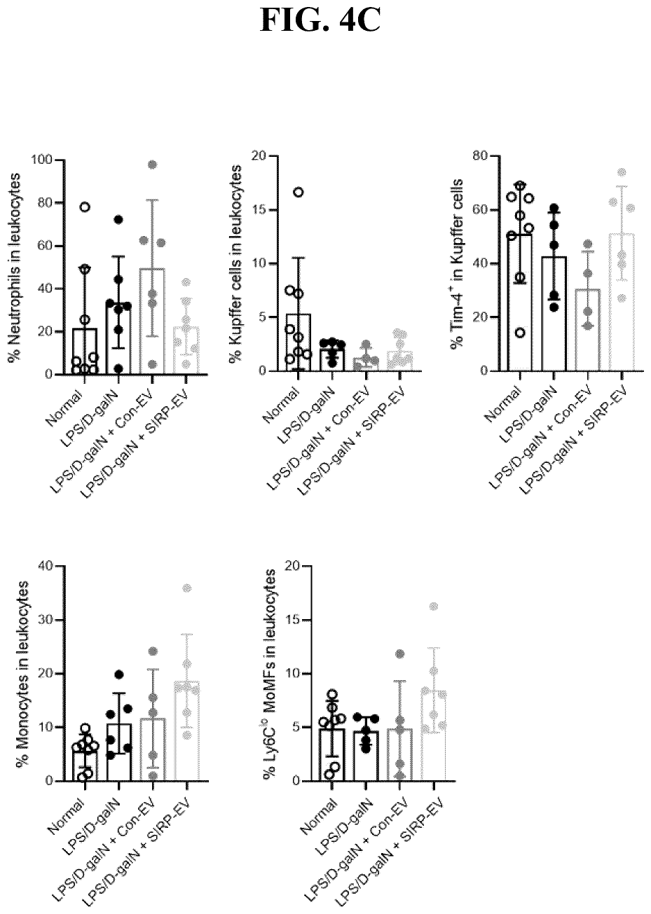

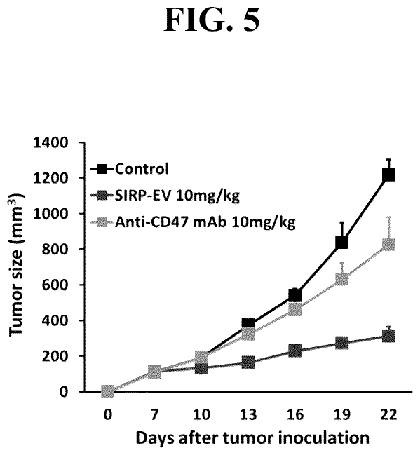

The efficacy of SIRP-EV was evaluated in an acute liver injury model, an acute inflammatory disease with CD47 overexpression, by injecting C57BL/6 6-week-old male mice with LPS (10 μg/kg) and D-galN (700 mg/kg) via IP injection. This model is a fast and severe acute liver injury model with about 50% mortality within 24 hours. Two hours before the induction, 30 μg of SIRP-EV was intravenously injected, and its efficacy was evaluated. The liver tissue was photographed six hours after the induction. As shown in FIG. 4A, the liver's appearance in the SIRP-EV treatment group was similar to that of a normal liver, compared to the liver that induced acute liver injury. Furthermore, it was confirmed that STRP-EV significantly increased survival rate compared to the Con-EV and the untreated group.

Six hours after inducing acute liver injury, blood was collected from the mice's vena cava, the serum was separated, and ALT and AST were analyzed, which are indicators of liver toxicity (DK Korea). Pro-inflammatory cytokines IL-6 and TNF-α present in the same serum were measured using ELISA (R&D Quantitative ELISA). As shown in FIG. 4B, SIRP-EV significantly reduced AST, ALT, IL-6, and TNF-α, which are liver toxicity-related numbers and pro-inflammatory cytokines, compared to Con-EV and the untreated control group.

At the same time point, six hours after inducing acute liver injury, liver tissue was extracted, and immune cell analysis was performed with the liver tissue. The separated liver tissue was singled out using a liver dissociation kit (Miltenyi) and gentleMACS tissue dissociator (Miltenyi). RBC lysis (Biolegend) was used to remove RBC present in the tissue. After sequential centrifugation, immune cells were isolated and markers of various immune cells present in the liver tissue were attached. The ratio of each immune cell per model was analyzed using a flow cytometer (Flow cytometry analysis, Beckman). Neutrophils, which dramatically increase in liver tissue after inducing acute liver injury, are well-known immune cells related to severe inflammation. As shown in FIG. 4C, the ratio of neutrophils within immune cells in the SIRP-EV treatment group normalized to the level of a healthy mouse liver.

Interestingly, Kupffer cells, which are responsible for removing lesions within the existing liver tissue, had a decreased ratio within the liver tissue in all groups induced with acute liver injury, unlike healthy mice. This is believed to be because the resident macrophages, Kupffer cells present in the liver tissue, were damaged and died due to the induction of acute liver injury. Interestingly, in the STRP-EV treated group, TIM4 expression related to dead/dying cell clearance increased compared to other control groups. In the SIRP-EV treatment group, the ratio of monocytes within immune cells was higher than in other groups, and the ratio of Ly6C^{lo} monocytes, which play a role in tissue repair, was also higher in the STRP-EV treatment group than in other groups.

These results imply that the rapid clearance of dying or dead cells in acute inflammatory disease tissues, facilitated by SIRP-EV, aids tissue regeneration. This mechanism not only activates Kupffer cells, which are responsible for lesion removal in existing liver tissue, but also swiftly infiltrates immune cells from the bloodstream into liver lesions, promoting resolution and repair.

### Example 5

### Superiority of SIRP-EV Over Competitive CD47 Blockade

The SIRPα protein is a membrane protein, and the surface of EV, which has the same membrane environment as the cell membrane, provides an optimal environment for the SIRPα protein to perform its inherent functions properly. Moreover, while other competitive CD47 blockades simply block CD47 signaling, SIRP-EV can remove the CD47 protein through endocytosis-mediated clearance beyond CD47 blockade.

Anti-cancer evaluation was conducted using a tumor model to verify whether SIRP-EV has higher efficacy than the existing CD47 blockade, the CD47 antibody.

10⁶ CT26.CL25 colon cancer cells were subcutaneously inoculated in the flank of 6-week-old Balb/c male mice. Seven days later, CD47 antibody (10 mg/kg) and SIRP-EV (10 mg/kg) were intravenously injected three times at three-day intervals. The experiment results show that SIRP-EV had much better anti-cancer efficacy than the CD47 antibody. This suggests that EV present a promising modality for the blockade of CD47.

### Example 6

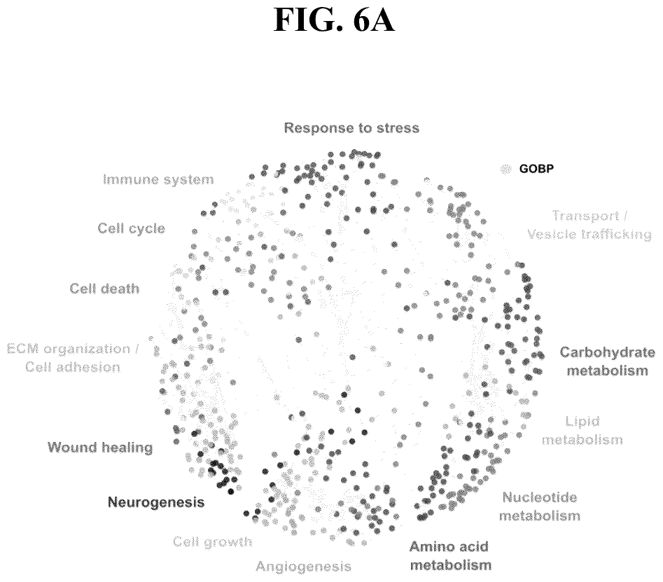

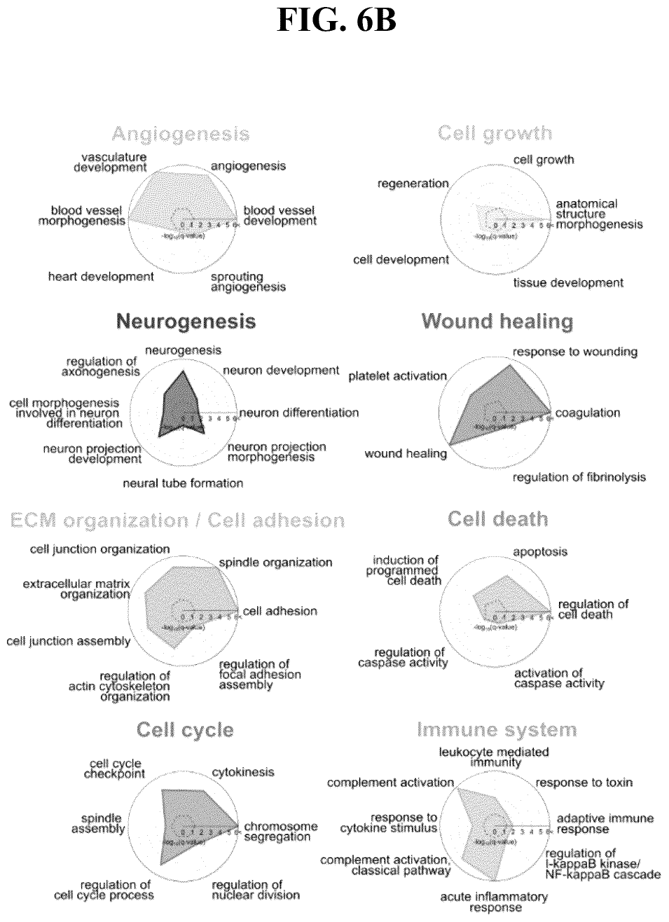

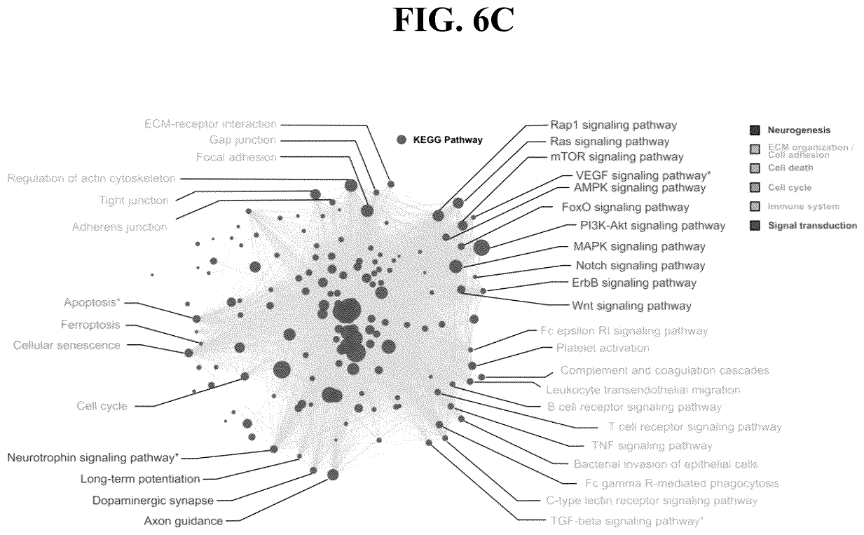

Regenerative Efficacy of Mesenchymal Stem Cell Derived SIRP-EV To verify whether regenerative functional factors of MSC are loaded within SIRP-EV derived from engineered MSC cells (_{MSC}SIRP-EV), a proteomic analysis was conducted. Using each 100 μg of EV in three replicate samples, LC-MS/MS analysis was carried out after labeling with the isobaric tag reagent, Tandem Mass Tag (TMT). A total of 7,448 proteins were identified, with correlation values of quantified proteins between and within batches being above 0.96, confirming the absence of batch-to-batch differences. To analyze groups of genes with similar functions and identify pathways enriched by Up-/Down-regulated genes, functional enrichment analysis was carried out using DEP (differentially expressed protein) quantification information (FIGS. 6A and 6B. Gene Ontology; FIGS. 6C and 6D. Pathway Analysis).

Referring to FIG. 6A, a GOBP (Gene Ontology Biological Process) network (where nodes are each GOBP term and edges are hierarchical relations between GOBP terms) was constructed using significantly over-represented (q-value <0.05) GOBP by 7,448 identified total proteins. The GOBP terms were differentiated into 14 GOBP categories and colored them differently. Many of the significant GOBP terms were related to the tropism and immunomodulation effects of MSCs (angiogenesis, cell growth, neurogenesis, wound healing, ECM organization/cell adhesion, cell death, cell cycle, immune system).

Referring to FIG. 6B, for the 8 GOBP categories related to the tropism and immunomodulation effects of MSC identified in FIG. 6A (angiogenesis, cell growth, neurogenesis, wound healing, ECM organization/cell adhesion, cell death, cell cycle, immune system), the significance level (i.e., q-value) of representative GOBP terms was plotted in a Radar Plot. Through GO analysis, functional annotation of STRP-EV, which appeared similar to the tropism of MSC cells, was made.

Referring to FIG. 6C, a pathway network (where nodes are each KEGG pathway and edges are the number of proteins shared between KEGG pathways) was constructed using significantly over-represented (q-value <0.05) KEGG (Kyoto Encyclopedia of Genes and Genomes) pathway by 7,448 identified total proteins. The KEGG pathways were differentiated into 6 pathway categories and colored them differently.

Referring to FIG. 6D, cellular network models were built for each representative pathway known to be involved in functions such as angiogenesis, neurogenesis, wound healing, and anti-inflammation, to highlight these functions related to the tropism and immunomodulation effects of MSC (VEGF signaling, neurotrophin signaling, apoptosis, TGF-beta signaling marked with asterisks in Figure C, respectively). The target genes with the KEGG DB were compared to check the pathways in which the genes were involved, and key proteins centered around the MSC function-related pathways were visualized.

### Example 7

### Superiority of Mesenchymal Stem Cell Derived SIRP-EV in Inflammatory Diseases

To elucidate the synergistic effect of SIRPα's immune therapeutic efficacy and the regenerative efficacy of stem cells, _{MSC}SIRP-EV was prepared. A lentiviral vector was used on human bone marrow-derived mesenchymal stem cells (MSC) to insert the gene that can express the SIRPα protein on the surface of EV, as indicated in the plasmid map of FIG. 1 for the SIRP-EV gene of interest and generated stable cells. Afterward, EV was extracted using the previously described EV isolation method. As a control group, EV derived from MSC (Naïve stem cell derived EV, _{M}scCon-EV) and SIRP-EV derived from HEK293T cells expressing the SIRPα protein through plasmid introduction (the same as used in the previous example) were used.

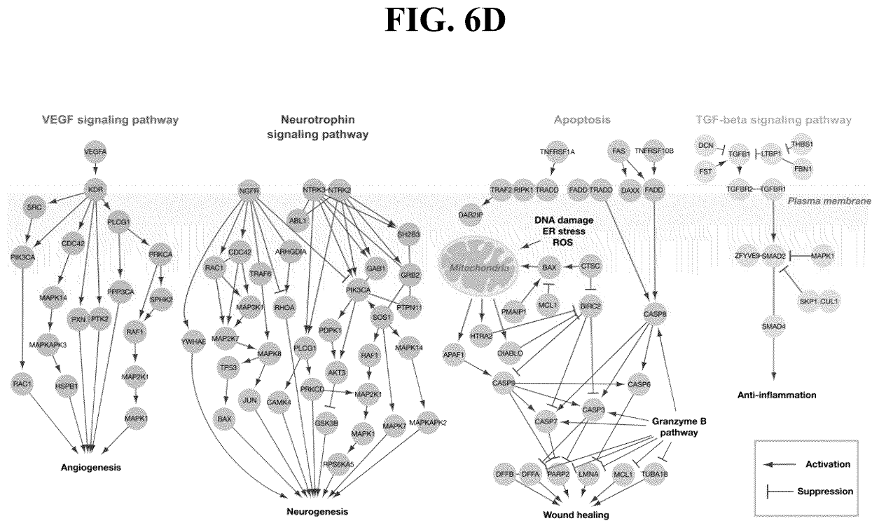

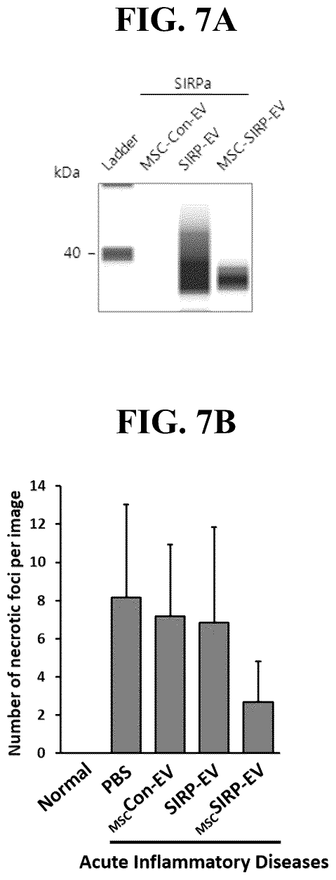

First, the expression of SIRPα in SIRP-EV derived from HEK293 cells and _{MSC}SIRP-EV derived from MSCs was confirmed by Western blot. Western blot samples were prepared by adding 5×SDS and PBS to make it 10 μg/10 μl. Western blot samples were incubated in a 95° C. heat block for 5 minutes. For the SDS gel, a gradient gel from Bio-Rad was used. The gel was run at 60 V for approximately an hour and a half, and the proteins on the SDS gel were transferred to an NC membrane using Bio-Rad's trans-blot turbo transfer system. After creating 5% skim milk with a TBST solution for 1-hour NC membrane blocking, SIRPα antibodies were added to the 5% skim milk and left overnight at 4° C. The solution was washed four times for 10 minutes each with a TBST solution. A rabbit secondary antibody (sigma) was added to 5% skim milk at a 1:3000 ratio and incubated at room temperature for 1 hour. Afterwards, it was washed four times for 10 minutes each with a TBST solution, and it was then photographed using Bio-Rad's ECL solution with a ChemiDoc imaging system at an exposure of 0.1 seconds. Even though they have the same SIRPα sequence, SIRP-EV showed higher SIRPα expression than MSCSIRP-EV as shown in FIG. 7A.

To evaluate the efficacy of _{MSC}SIRP-EV, an acute liver injury model induced by carbon tetrachloride (CCl4) was used. Specifically, CCl4 (289116, Sigma Aldrich, Darmstadt, Germany) was intraperitoneally injected into 6-week-old C57BL/6 mice using corn oil (C8265, Sigma Aldrich, Darmstadt, Germany) to make 30% CCl4. 24 hours after CCL4 injection, liver tissue was extracted to make paraffin blocks for efficacy analysis. Paraffin slides were then made from this block, and Hematoxylin and Eosin staining (H&E staining) was performed. Specifically, the H&E staining was performed in the order of dewaxing, dehydration, hematoxylin, differentiator, bluing, eosin, dehydration, clearing, and cover-slipping. Then, random samples were captured at 200× magnification using an optical microscope, securing data with 30 shots per sample. The necrotic foci were manually counted in each picture to derive quantitative data to measure the severity of acute liver injury.

As shown in FIG. 7B, even though SIRPα expression was higher in SIRP-EV, the number of necrotic foci in the liver tissue where acute liver injury was induced was significantly reduced in _{MSC}SIRP-EV compared to the control group, _{MSC}Con-EV, and SIRP-EV derived from HEK293T cells. This suggests that the regenerative efficacy of stem cells and the immunotherapeutic efficacy of SIRPα in removing pathological cells by immune cells can synergistically restore liver tissue damage caused by acute inflammatory diseases.

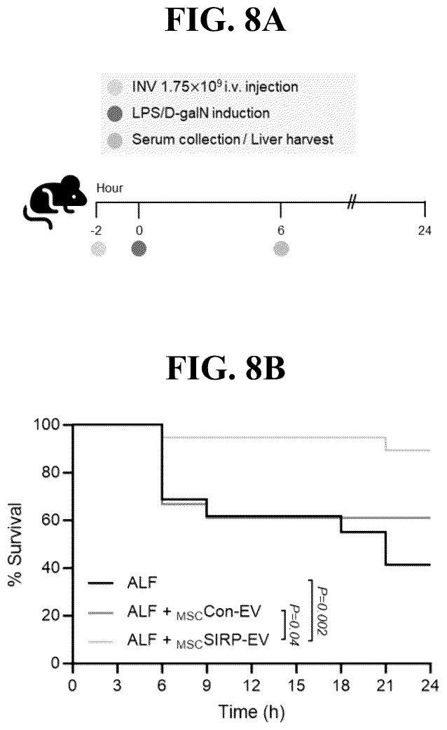

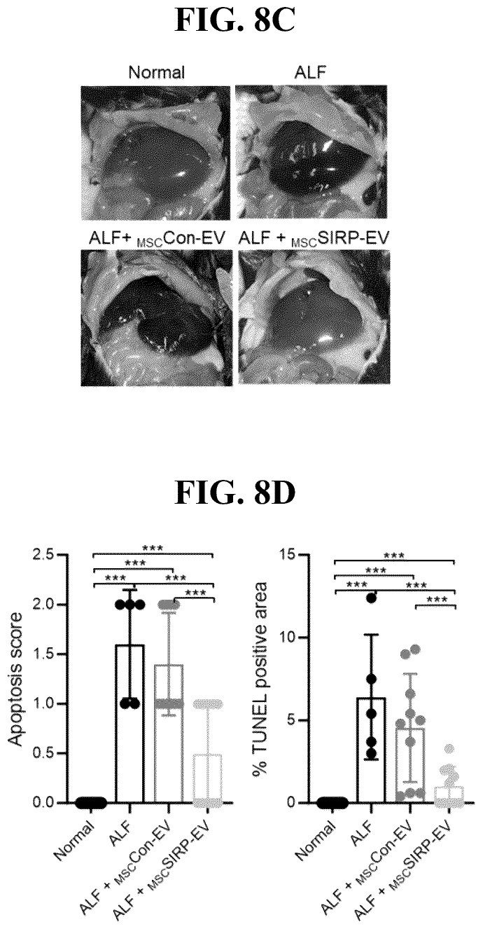

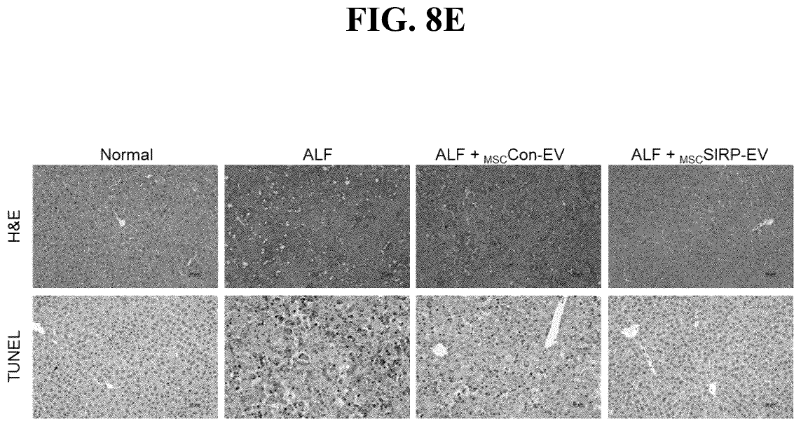

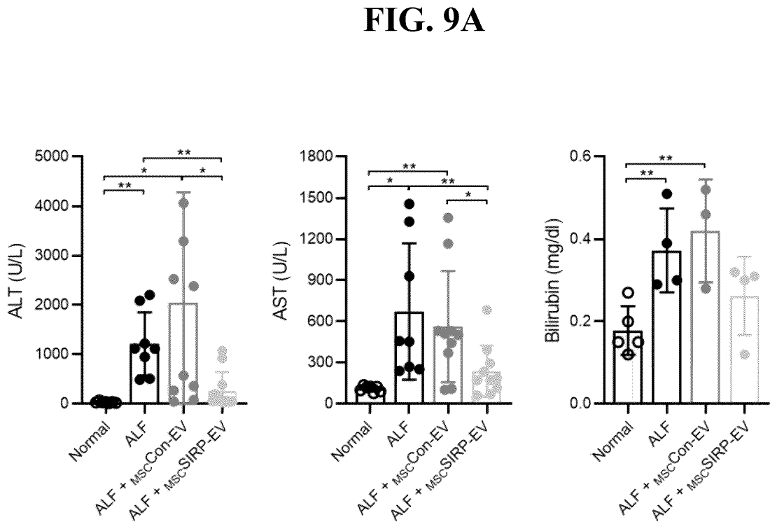

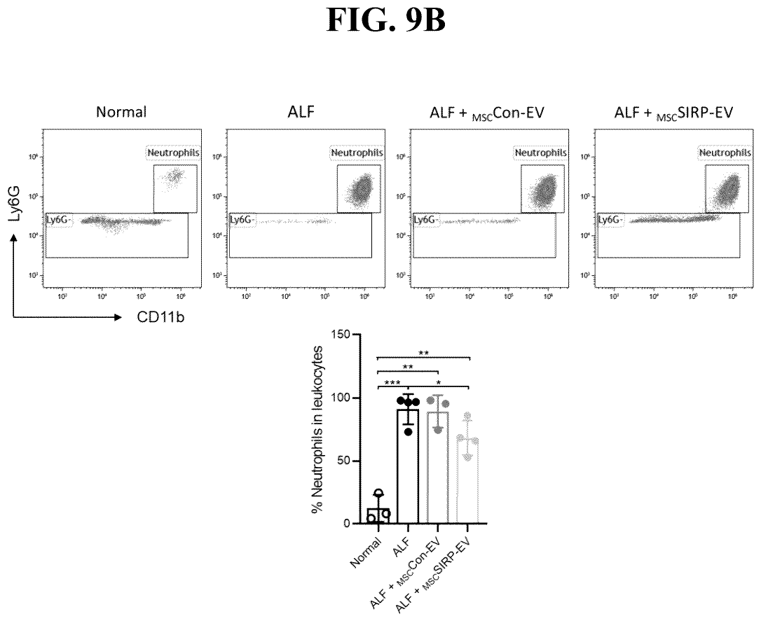

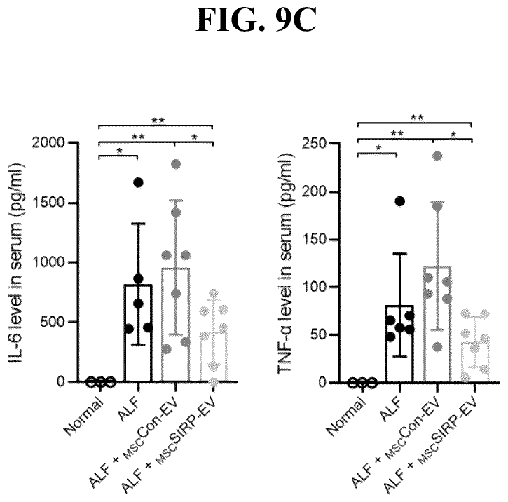

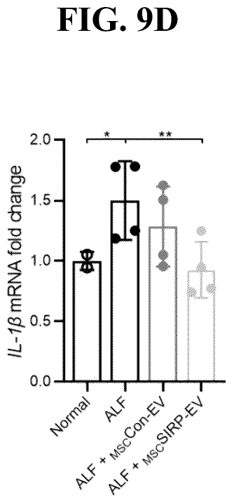

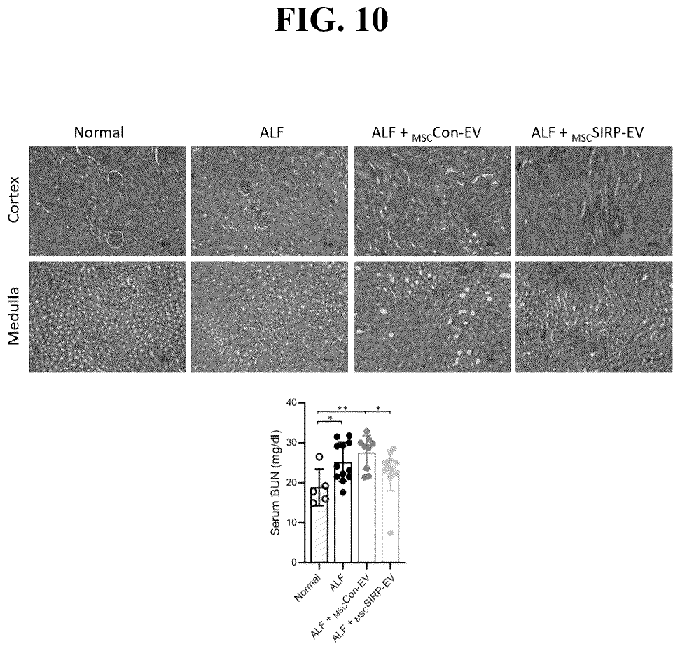

The efficacy of _{MSC}SIRP-EV was evaluated in an acute liver injury model, an acute inflammatory disease with overexpression of CD47, by injecting C57BL/6 6-week-old male mice with LPS (10 μg/kg) and D-galN (700 mg/kg) via IP injection. Even though _{MSC}SIRP-EV (about 2 μg, 1.75×10⁹) was injected IV at less than one twentieth of the therapeutic concentration of SIRP-EV (30 μg) in the same model (FIG. 8A), it showed a very promising improvement in survival rate compared to the control group, _{M}scCon-EV, in this acute liver injury model (FIG. 8B). Upon visual inspection, the liver with acute liver injury after MSCSIRP-EV treatment was observed to have a similar shape to a normal liver (FIG. 8C). H&E and TUNEL assays also demonstrated that _{MSC}SIRP-EV effectively removed dead cells present in acute liver injury tissue (FIGS. 8D and 8E). Blood tests confirmed that _{MSC}SIRP-EV injection in the acute liver injury model significantly reduced liver toxicity indicators ALT, AST, bilirubin, and decreased the proportion of neutrophils that worsen inflammation in the liver tissue (FIGS. 9A and 9B). Additionally, it was confirmed through ELISA and RT-PCR that _{MSC}SIRP-EV treatment effectively suppressed inflammatory cytokines IL-6, TNF-α, and IL-1β (FIGS. 9C and 9D). Furthermore, in this model, toxicity can also occur in the kidneys, but _{MSC}SIRP-EV treatment normalized kidney tissue as confirmed by H&E staining, and significantly reduced blood urea nitrogen (BUN) levels related to kidney function in the blood, as determined statistically (FIG. 10).

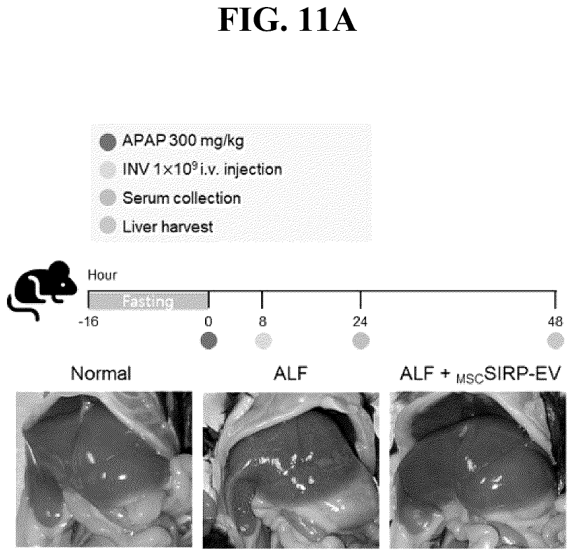

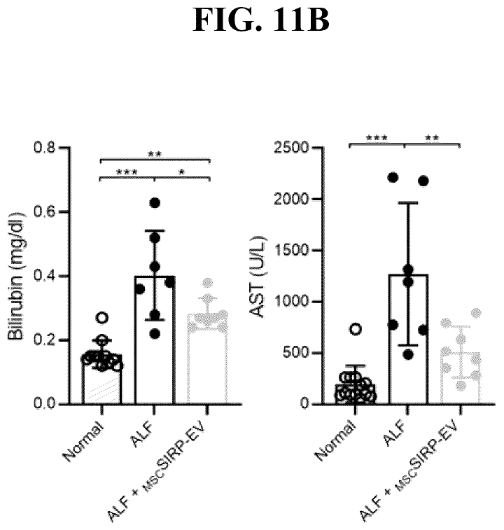

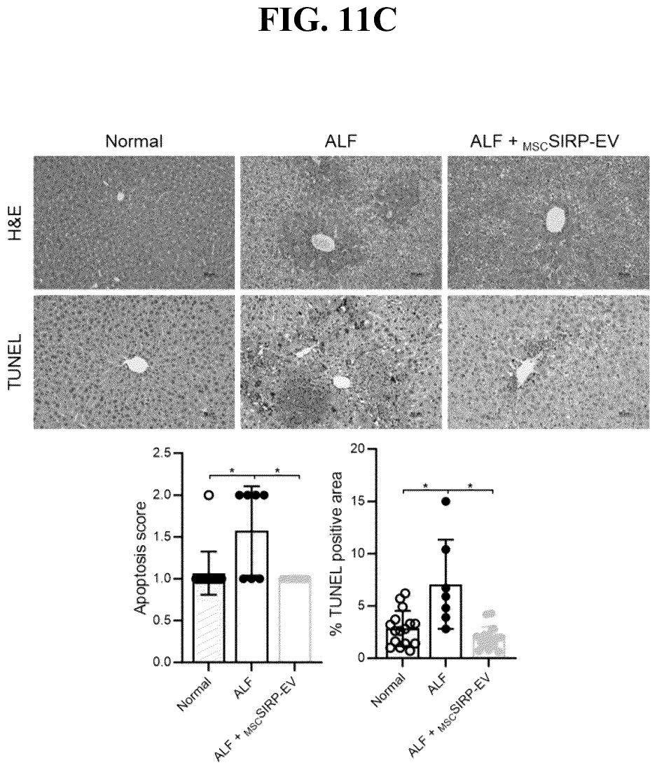

To verify the broad application of _{MSC}SIRP-EV in an acute inflammation model, 1×10⁹ _{MSC}SIRP-EV was IV-injected eight hours after APAP injection, where CD47 overexpression was observed, in the APAP induced acute liver injury model. The results showed that visually, _{MSC}SIRP-EV treatment made the liver tissue with acute injury appear similar to normal liver tissue (FIG. 11A), and significantly reduced bilirubin and AST related to liver toxicity (FIG. 11i). Furthermore, it was clarified through H&E and TUNEL assays that _{MSC}SIRP-EV injection very effectively removed dead/dying cells from liver tissue with injury in the acute liver injury situation compared to the untreated control group (FIG. 11C). These results suggest that _{MSC}SIRP-EV exhibited significant therapeutic effects in inflammatory diseases with CD47 overexpression.

In summary, _{MSC}SIRP-EV synergizes the regenerative potential inherited from stem cells and the pathogenic cell removal effect of the expressed SIRPα. Specifically in inflammatory diseases, _{MSC}SIRP-EV induces a stronger therapeutic effect compared to _{MSC}Con-EV and HEK293 cell derived SIRP-EVs.

1. A method for preventing or treating cancer or inflammatory disease, condition, or symptom, the method comprising administering to a subject in need thereof a prophylactically or therapeutically effective amount of stem cell-derived extracellular vesicles (EV) that comprise a signal regulatory protein (SIRP), a fragment of the SIRP, a variant of the SIRP, a fragment of the variant, or a variant of the fragment.

2. The method of claim 1, wherein the SIRP is SIRPα, SIRP7, or both.

3. The method of claim 1, wherein the stem cells are embryonic stem cells or adult stem cells.

4. The method of claim 3, wherein the adult stem cells are selected from the group consisting of mesenchymal stem cells, human tissue-derived mesenchymal stromal cells, human tissue-derived mesenchymal stem cells, multipotent stem cells, and amniotic epithelial cells.

5. The method of claim 4, wherein the mesenchymal stem cells are derived from one or more tissues selected from the group consisting of umbilical cord, cord blood, bone marrow, fat, muscle, nerve, skin, amnion, and placenta.

6. The method of claim 1, wherein the SIRP, the fragment, or the variant is linked to at least one EV protein.

7. The method of claim 6, wherein the EV protein is selected from the group consisting of CD9, CD53, CD63, CD81, CD54, CD50, FLOT1, FLOT2, CD49d, CD71, CD133, CD138, CD235a, ALIX, syntenin-1, syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, tetraspanin, Fc receptor, interleukin receptor, immunoglobulin, MHC-I components, MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LlCAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, PDGFR, GPI anchor protein, lactadherin, syndecan, synaptotagmin, apoptosis-linked gene 2-interacting protein X (ALIX), PTGFRN, a fragment thereof, a variant thereof, a variant of the fragment and a fragment of the variant.

8. The method of claim 1, wherein the cancer or the inflammatory disease, condition, or symptom is related to CD47 positive and/or CD47 overexpression.

9. The method of claim 1, wherein the inflammatory disease, condition, or symptom is selected from the group consisting of single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, dermatologic inflammation, gastrointestinal inflammation, tendinopathies, allergy, asthma, glomerulonephritis, pancreatitis, hepatitis, non-alcoholic steatohepatitis (NASH), gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, crohn's and ulcerative colitis, terminal ileitis, ophthalmic inflammation, retinal degeneration, retinal detachment, retinitis pigmentosa, inherited retinal diseases, age-related macular degeneration, glaucoma, inflammatory arthritis, rheumatoid arthritis, osteoarthritis, alcoholic steatohepatitis, hepatotoxicity, liver infection, remote liver injury, lupus, autoimmune diseases associated with acute or chronic inflammation, and acute or chronic inflammation associated with viral, bacterial or fungal infection.

10. The method of claim 9, wherein the organ failure is selected from the group consisting of acute liver failure, bone marrow failure, acute kidney failure, and acute heart failure.

11. The method of claim 9, wherein the viral infection is selected from the group consisting of hepatitis virus infection, ZIKA virus infection, herpes virus infection, papillomavirus infection, influenza virus infection, coronavirus infection, COVID-19, and SARS.

12. The method of claim 9, wherein the fibrosis is selected from the group consisting of pulmonary fibrosis, cystic fibrosis, idiopathic pulmonary fibrosis, skin fibrosis, kidney fibrosis, bone marrow fibrosis, interstitial pulmonary fibrosis, liver fibrosis, bridging fibrosis of the liver, arthrofibrosis, keloid fibrosis, mediastinal fibrosis, myelofibrosis, myocardial fibrosis, nephrogenic systemic fibrosis, progressive massive fibrosis, retroperitoneal fibrosis, and stromal fibrosis.

13. The method of claim 1, wherein the cancer is selected from the group consisting of melanoma, renal cancer, prostate cancer, breast cancer, colon cancer, rectum adenocarcinoma, lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumor of childhood, lymphocytic lymphoma, cancer of the bladder, cancer of the kidney or ureter, carcinoma of the renal pelvis, neoplasm of the central nervous system (CNS), non-small cell lung cancer (NSCLC), primary CNS lymphoma, tumor angiogenesis, spinal axis tumor, brain stem glioma, glioblastoma multiforme, low-grade gliomas, pituitary adenoma, Kaposi's sarcoma, epidermoid cancer, squamous cell cancer, T-cell lymphoma, B-cell lymphomas, cholangiocarcinoma, thymoma, adrenocortical cancer, cervical cancer, endocervical cancer, myxofibrosarcoma, undifferentiated pleomorphic sarcoma, dedifferentiated liposarcoma, dysembryoplastic neuroepithelial tumor, ependymoma, nasopharyngeal carcinoma, choroid plexus carcinoma, myoepithelial carcinoma, alveolar rhabdomyosarcoma, rhabdomyosarcoma, atypical teratoid/mabdoid tumor, desmoplastic small round cell tumor, fibromatosis, synovial sarcoma, wilms tumor, myofibromatosis, ewing sarcoma, infantile fibrosarcoma, INI-deficient soft tissue sarcoma, medulloblastoma and environmentally induced cancer.

14. The method of claim 1, wherein the extracellular vesicle comprises an anti-cancer agent, an anti-inflammatory agent, or both.


SEQUENCE LISTING (10 sequences)

SEQ ID NO: 1 - protein, 118 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKP

SEQ ID NO: 2 - protein, 117 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKP

SEQ ID NO: 3 - protein, 117 aa, synthetic construct
EEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKP

SEQ ID NO: 4 - protein, 118 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKP

SEQ ID NO: 5 - protein, 117 aa, synthetic construct
EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRAKP

SEQ ID NO: 6 - protein, 117 aa, synthetic construct
EEELQIIQPDKSVLVAAGETATLRCTITSLFPVGPIQWFRGAGPARVLIYNQRQGPFPRVTTVSETTKRENMDFSISISNITPADAGTYYCIKFRKGSPDTEFKSGAGTELSVRAKP

SEQ ID NO: 7 - protein, 118 aa, synthetic construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKP

SEQ ID NO: 8 - protein, 118 aa, synthetic construct
EEELQIIQPEKLLLVTVGKTATLHCTVTSLFPVGPVLWFRGVGPGRVLIYNQRQGPFPRVTTVSDTTKRNNMDFSIRISSITPADVGTYYCIKFRKGSPENVEFKSGPGTEMALGAKP

SEQ ID NO: 9 - protein, 118 aa, synthetic construct
EEELQIIQPEKLLLVTVGKTATLHCTITSLFPVGPVLWFRGVGPGRVLIYNQRQGPFPRVTTVSDTTKRNNMDFSIRISSITPADVGTYYCIKFRKGSPENVEFKSGPGTEMALGAKP

SEQ ID NO: 10 - protein, 169 aa, synthetic construct
MMLQHLVIFCLGLVVQNFCSPGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPEFKYPLLIGIGLSAVIGLLSCLIGYCSS